SEQUENCE LISTING (1 sequence)

SEQ ID NO: 1 - protein, 164 aa, Artificial Sequence
  synthesized sequence
GGKSGGNKKSDGVKESSESTNTTIEDEDTKVRKQEIIKVTEQLIEAISNGDFESYTKMCDPGMTAFEPEALGNLVEGLDFHRFYFENLWSRNSKPVHTTILNPHIHLMGDESACIAYIRITQYLDAGGIPRTAQSEETRVWHRRDGKWQIVHFHRSGAPSVLPH

(19) United States
(12) Patent Application Publication
(10) Pub. No.: US 2014/0255314 A1
(43) Pub. Date: Sep. 11, 2014

(54) METHOD FOR INDUCING PHYSIOLOGICAL ADJUSTMENT USING HIGH DENSITY DISPLAY OF MATERIAL

(71) Applicant: Tae Kook Kim, Gyeonggi-do (KR)

(72) Inventor: Tae Kook Kim, Gyeonggi-do (KR)

(21) Appl. No.: 14/237,832

(22) PCT Filed: Aug. 10, 2012
(86) PCT No.: PCT/KR2012/006368

§ 371 Date: Apr. 21, 2014

(30) Foreign Application Priority Data
Aug. 10, 2011 (KR) 10-2011-0079887

(51) Int. Cl. G01N 33/58; A61K 47/48; G01N 33/50; A61B 5/00; A61N 1/32

(52) U.S. Cl. CPC G01N 33/582; A61B 5/0071; A61N 1/327; G01N 33/5023; A61K 47/48246
USPC 424/9.6; 600/431; 604/501; 435/375; 435/6.13; 424/94.5; 514/21.2; 424/85.1; 435/7.92

(57) ABSTRACT

The present invention relates to a method of regulating or inducing specific physiological conditions or functions using one or more materials displayed on a nano-assembly matrix at high density in cells or in vivo. Specifically, the present invention relates to a method of effectively inducing specific physiological regulation in cells or in vivo by the high-density display of bioactive materials. According to the method of the invention, physiological regulation in cells or in vivo can be optionally induced by regulating the assembly and disassembly of nano assembly (unit) matrix or the display or trapping of specific materials on nano assembly (unit) matrix.

### TECHNICAL FIELD

The present invention relates to a method of regulating or inducing specific physiological conditions or functions using one or more materials displayed on a nano-assembly matrix at high density in cells or in vivo. Specifically, the present invention relates to a method of effectively inducing specific physiological regulation in cells or in vivo by the high-density display of bioactive materials.

### BACKGROUND ART

In general, various physiological functions are regulated by the dynamic interactions between various bioactive molecules. If such interactions do not occur properly, problems arise to cause diseases. For example, proteins in vivo perform their functions by interaction with other proteins. Generally, two proteins having complementary structures interact with each other, and a bioactive compound interacts specifically with the specific portion of the three-dimensional protein structure. Generally, the interaction between two proteins strongly implies that they are functionally related. Furthermore, a bioactive compound interacting specifically with the specific portion of a disease-associated protein has potential as a therapeutic agent which can diagnose, prevent, treat or alleviate the disease by controlling the activity of the protein.

Accordingly, in the field of new drug development, there have been studies on various methods of detecting novel target proteins or screening bioactive molecules as drug candidates by detecting the interaction between a “bait” whose function and feature are known and a “prey” which is an interaction partner to be analyzed and detected. Thus, the identification and isolation of a novel target protein through the analysis of the interaction between bioactive molecules are considered as very important research projects for obtaining useful information about the activity, effectiveness and adverse effects of bioactive drugs. Additionally, target proteins promote the understanding of biological pathways and signal transduction systems and provide information on fundamental cellular regulation and disease mechanisms. Such information is a very powerful tool for developing new drugs, improving existing drugs and discovering the novel pharmaceutical use of existing drugs by analyzing and detecting bioactive compounds which interact with the target proteins.

Modern medicine faces the challenge of developing safer and more effective therapies against various human diseases. However, many drugs currently in use are prescribed by the biological effects in disease models without their target proteins and molecular targets (Burdine, L. et al., Chem. Biol. 11: 593, 2004). Bioactive natural products are an important source for drug development, but their modes of action are usually unknown (Clardy, J. et al., Nature 432:829, 2004). Elucidation of their physiological targets and molecular targets is essential for understanding their therapeutic and adverse effects, thereby enabling the development of improved second-generation therapeutics. Moreover, the discovery of novel targets of clinically proven compounds may suggest new therapeutic applications (Ashburn, T. T. et al., Drug Discov. 3:673, 2004).

In chemical and biological field employing cell-based screening, “target screening” is used to identify small molecules with a desired phenotype from large compound libraries (Strausberg, R. L. at al., Science 300:294, 2003; Stockwell, B. R. Nature 432:846, 2004). Despite the great benefits of such screening, this approach has been hampered by the daunting task of target identification. However, the development of such identification technology is very important in various bioscience fields, including genomics, proteomics and system biology, because effective detection of diverse intracellular molecular interactions, including protein (or small molecule)-protein, is essential for understanding dynamic biological processes and regulatory networks.

In the field of target screening, several technologies, including affinity chromatography (Phizicky, E. M. et al., Microbiol. Rev. 59:94, 1995; Mendelsohn, A. R. et al., Science 284:1948, 1999), protein-small molecule microarray, phage display (Sche, P. P. et al., Chem. Biol. 6:707, 1999), yeast two-/three-hybrid assay (Licitra, E. J. et al., Proc. Natl. Acad. Sci. USA 93:12817, 1996), expression profiling, and parallel analysis (Zheng, O. et al., Chem. Biol. 11: 609, 2004) of yeast strains with heterologous deletions, have been utilized to analyze interactions between bioactive molecules.

However, such technologies all suffer from diverse problems, including high background, false positives, low sensitivity, inappropriate folding after protein expression, indirectness, lack of modification after protein expression, or limited target accessibility including cellular compatibility. In addition, the use of artificial experimental milieu, such as in vitro binding conditions or non-mammalian cells, sometimes causes errors in experimental results.

Accordingly, it is most preferable to directly examine the interaction between bioactive molecules in a state in which high sensitivity and selectivity were considered in physiological or pharmaceutical terms. Thus, it is considered that it is very important to develop the above-described base technology in order to offer various advantages over the prior art.

First, by probing the interactions between physiologically or pharmaceutically relevant bioactive materials and molecules, misleading outcomes produced by an artificial experimental setting can be greatly diminished. Second, it is possible to directly translate the interaction between bioactive molecules into a clear readout signal, unlike indirect readout methods that are dependent on overall expression profiles or complex biological phenotypes. Thus, intrinsic false positives/negatives or error-prone deductions about bioactive molecules and molecular targets can be obviated. Third, it is possible to perform dynamic, single-cell analysis for the interactions between bioactive materials and molecules. Dynamic analysis of individual living cells provides an effective method which can analyze intracellular processes occurring non-simultaneously among heterogeneous cells, over a broad range in physiological and pharmaceutical terms.

Therefore, the above-described base technology can be used to detect a variety of biological interactions between bioactive materials and molecules (e.g., interaction between a bioactive small molecule and a protein) and protein modifications (e.g., phosphorylation) within living cells in a broad range of tissues and disease states, but have many limitations. Thus, the development of new base technology is required.

Accordingly, the present inventors previously studied a method for dynamically analyzing the interactions between bioactive molecules not only in vivo, but also in vitro, which overcomes the above-described problems occurring in the prior art. As a result, the present inventors found that the interactions between various bioactive molecules can be analyzed and detected by analyzing whether the interactions between the bioactive molecules result in the formation of a nano-assembly matrix or the co-localization of these molecules on the nano-assembly matrix (see Korean Patent Application No. 10-2008-0079957).

Meanwhile, various physiological (assembly) matrices are present as signal some in cells and as “-some” or “complex” such as exosome in an extracellular environment in vivo. It is known that bioactive materials, including one or more relevant proteins, are present in such physiological assembly matrices, and thus specific physiological functions in cells or in vivo are effectively regulated. It was found that multi/poly-valent interactions play a very important role in most physiological regulations, like in efficient physiological regulation mediated by such matrices (Mammen, M. et al., Angew. Chem. Int. Ed. 37:2755, 1998; Kiessling, L. L. et al., Angew. Chem. Int. Ed. 45:2348, 2006). In other words, multi/poly-valent interactions mainly play an important role in the interactions between most bioactive materials, including proteins, compared to mono-valent interactions.

Accordingly, based on the characteristics and interactions of such physiological matrices, the present inventors have understood that when one or more kinds of bioactive molecules (materials) displayed on a nano-assembly matrix at high density are present at high concentrations, the local concentration thereof in cells or in vivo is increased, and their multi/poly-valent interactions with the targets or the like are effectively induced, and thus they function synergistically with each other, and when these bioactive molecules are functionally coordinated, physiological functions in cells or in vivo can be effectively regulated and induced. Based on this understanding, the present inventors have conducted extensive experiments and studies, and as a result, have found that, when bioactive materials are displayed on an artificially formed and induced nano-assembly matrix at high density, physiological functions in cells or in vivo can be effectively regulated and induced, thereby completing the present invention. In addition, the present inventors have found that physiological functions in cells or in vivo can be optionally regulated and induced by artificially regulating and inducing the assembly and disassembly of the nano-assembly matrix or the display or trapping of specific materials on the nano-assembly matrix.

The information disclosed in the Background Art section is only for the enhancement of understanding of the background of the present invention, and therefore may not contain information that forms a prior art that would already be known to a person skilled in the art.

### DISCLOSURE OF INVENTION

### Technical Problem

It is an object of the present invention to provide a method of artificially regulating and inducing physiological functions in cells or in vivo, which are mediated by a specific bioactive molecule or material. Specifically, an object of the present invention is to effectively induce the regulation of physiological functions either by bioactive materials displayed on an artificial nano-assembly matrix at high density or by the interaction of the displayed materials with other relevant materials.

According to their kinds and physiological functions, molecules or materials displayed at high density, for example, antibodies, antigens, epitopes, viral proteins and peptides (e.g., HIV Tat, HBV and SARS proteins and peptides), disease cell-specific receptor or marker protein-targeting proteins/peptides, therapeutic receptor-binding proteins/peptides, therapeutic proteins/peptides, hemoglobin, Gd3+ ions, therapeutic drugs, silver condensing peptides, metal scavenging peptides, etc., can be effectively used for the development of vaccines, therapeutics, diagnostics, imaging agents, metal chelating agents, blood substitutes, gelling agents, purification platforms, drug delivery platforms, etc.

Such molecules or materials displayed at high density can be administered to cells, tissues or living bodies in the form of various bioactive molecules, including genes, proteins and compounds, and the formation and disassembly of the nano-assembly matrix can be optionally regulated in vitro or in vivo, for example, inside or outsides cells or living bodies.

### Technical Solution

To achieve the above objects, in one aspect, the present invention provides a method for regulating or inducing physiological conditions or functions in cells or in vivo (FIG. 2), the method comprising the steps:

(i) providing mediator (regulator) materials, detector materials and a nano-assembly matrix-forming material to the same field or system; and

(ii) forming a nano-assembly matrix by the interaction between the mediator (regulator) materials to display the detector materials at high density, thereby regulating or inducing physiological conditions or functions that are mediated by the mediator (regulator) materials or the detector materials.

The present invention also provides a method for regulating or inducing physiological conditions or functions in cells or in vivo, the method comprising the steps of:

(i) providing first mediator (regulator) materials, second mediator (regulator) materials, detector materials and a nano-assembly matrix-forming material to the same field or system;

(ii) forming a nano-assembly matrix by the interaction between the first mediator (regulator) materials, and displaying the detector materials at high density by the interaction between the second mediator (regulator) materials, thereby regulating or inducing physiological conditions or functions, which are mediated by the mediator (regulator) materials or the detector materials.

The present invention also provides a method for regulating or inducing physiological conditions or functions in cells or in vivo, the method comprising the steps of:

(i) providing detector materials and a nano-assembly matrix-forming material to the same field or system; and

(ii) displaying the detector materials on nano-matrices at high density, thereby regulating or inducing physiological conditions or functions that are mediated by the detector materials.

The present invention also provides a method for regulating or inducing physiological conditions or functions in cells or in vivo, the method comprising the steps of:

(i) providing detector materials and nano-assembly matrix-forming materials to the same field or system; and

(ii) forming a nano-assembly matrix by the interaction between the detector materials to display the detector materials at high density, thereby regulating or inducing physiological conditions or functions that are mediated by the detector materials.

The present invention also provides a composition for vaccination, prevention, material delivery or treatment against disease related to physiological conditions or functions in cells or in vivo, the composition comprising a nano-assembly matrix isolated by a method comprising the steps of:

(i) providing mediator (regulator) materials, detector materials and a nano-assembly matrix-forming material to the same field or system; and

(ii) forming a nano-assembly matrix by the interaction between the mediator (regulator) materials to display the detector materials at high density, and isolating the formed nano-assembly matrix.

The present invention also provides a composition for vaccination, prevention, material delivery or treatment against disease related to physiological conditions or functions in cells or in vivo, the composition comprising a nano-assembly matrix isolated by a method comprising the steps of:

(i) providing first mediator (regulator) materials, second mediator (regulator) materials, detector materials and a nano-assembly matrix-forming material to the same field or system; and

(ii) forming a nano-assembly matrix by the interaction between the first mediator (regulator) materials, displaying the detector materials at high density by the interaction between the second mediator (regulator) materials, and isolating the formed nano-assembly matrix.

The present invention also provides a composition for vaccination, prevention, material delivery or treatment against disease related to physiological conditions or functions in cells or in vivo, the composition comprising a nano-assembly matrix isolated by a method comprising the steps of:

(i) providing detector materials and a nano-assembly matrix-forming material to the same field or system; and

(ii) displaying the detector materials on nano-matrices at high density, and isolating the formed nano-matrices.

The present invention also provides a composition for vaccination, prevention, material delivery or treatment against disease related to physiological conditions or functions in cells or in vivo, the composition comprising a nano-assembly matrix isolated by a method comprising the steps of:

(i) providing mediator (regulator) materials, detector materials and a nano-assembly matrix to the same field or system; and

(ii) displaying the mediator materials and the detector materials on nano-matrices at high density, and isolating the formed nano-matrices.

The present invention also provides a composition for vaccination, prevention, material delivery or treatment against disease related to physiological conditions or functions in cells or in vivo, the composition comprising a nano-assembly matrix isolated by a method comprising the steps of:

(i) providing mediator (regulator) materials and a nano-assembly matrix-forming material to the same field or system; and

(ii) displaying the mediator (regulator) materials on nano-matrices, and isolating the formed nano-matrices.

The present invention also provides a composition for vaccination, prevention, material delivery or treatment against disease related to physiological conditions or functions in cells or in vivo, the composition comprising a nano-assembly matrix isolated by a method comprising the steps of:

(i) providing detector materials and a nano-assembly matrix-forming material to the same field or system; and

(ii) forming a nano-assembly matrix by the interaction between the detector materials to display the detector materials at high density, and isolating the formed nano-assembly matrix.

The present invention also provides a method for screening a material that regulates or induces physiological conditions or functions in cells or in vivo, the method comprising the steps of:

(i) providing mediator (regulator) materials, detector materials and a nano-assembly matrix-forming material to the same field or system;

(ii) forming a nano-assembly matrix by the interaction between the mediator (regulator) materials to display the detector materials at high density;

(iii) providing target candidates to the nano-assembly matrix; and

(iv) selecting, as the material that regulates or induces physiological conditions or functions in cells or in vivo, a target candidate corresponding to a case in which physiological conditions or functions in the presence of the target candidate change compared to physiological conditions or functions in the absence of the target candidate.

The present invention also provides a method for screening a material that regulates or induces physiological conditions or functions in cells or in vivo, the method comprising the steps of:

(i) providing first mediator (regulator) materials, second mediator (regulator) materials, detector materials and a nano-assembly matrix-forming material to the same field or system;

(ii) forming a nano-assembly matrix by the interaction between the first mediator (regulator) materials, and displaying the detector materials at high density by the interaction between the second mediator (regulator) materials;

(iii) providing target candidates to the nano-assembly matrix; and

(iv) selecting, as the material that regulates or induces physiological conditions or functions in cells or in vivo, a target candidate corresponding to a case in which physiological conditions or functions in the presence of the target candidate change compared to physiological conditions or functions in the absence of the target candidate.

The present invention also provides a method for screening a material that regulating or inducing physiological conditions or functions in cells or in vivo, the method comprising the steps of:

(i) providing detector materials and a nano-assembly matrix-forming material to the same field or system; and

(ii) displaying the detector materials on nano-matrices at high density;

(iii) either providing target candidates to the nano-matrices, or isolating the nano-matrices, introducing the isolated nano-matrices into a cell, a tissue or a living body, and providing target candidates to a nano-assembly matrix formed by the interaction between the detector materials or by added mediator (regulator) materials; and

(iv) selecting, as the material that regulating or inducing physiological conditions or functions in cells or in vivo, a target candidate corresponding to a case in which physiological conditions or functions in the presence of the target candidate change compared to physiological conditions or functions in the absence of the target candidate change.

The present invention also provides a method for screening a material that regulates or induces physiological conditions or functions in cells or in vivo, the method comprising the steps of:

(i) providing first mediator (regulator) materials, detector materials and a nano-assembly matrix-forming material to the same field or system;

(ii) displaying the first mediator (regulator) materials and the detector materials on nano-matrices at high density;

(iii) either providing target candidates to the nano-matrices, or isolating the nano-matrices, introducing the isolated the nano-matrices into a cell, a tissue or a living body, and providing target candidates to a nano-assembly matrix formed by the interaction between the first (mediator) materials or by added second mediator (regulator) materials; and

(iv) selecting, as the material that regulating or inducing physiological conditions or functions in cells or in vivo, a target candidate corresponding to a case in which physiological conditions or functions in the presence of the target candidate change compared to physiological conditions or functions in the absence of the target candidate change.

The present invention also provides a method for screening a material that regulates or induces physiological conditions or functions in the cells or in vivo, the method comprising the steps of:

(i) providing detector materials and nano-assembly matrix-forming materials to the same field or system;

(ii) forming a nano-assembly matrix by the interaction between the detector materials to display the detector materials at high density;

(iii) providing target candidates to the nano-assembly matrix; and

(iv) selecting, as the material that regulating or inducing physiological conditions or functions in cells or in vivo, a target candidate corresponding to a case in which physiological conditions or functions in the presence of the target candidate change compared to physiological conditions or functions in the absence of the target candidate change.

The present invention also provides a method for diagnosing, preventing or treating disease related to physiological conditions or functions in cells or in vivo, the method comprising the steps of:

(i) providing mediator (regulator) materials, detector materials and a nano-assembly matrix-forming material to the same field or system;

(ii) forming a nano-assembly matrix by the interaction between the mediator (regulator) materials to display the detector materials at high density, and isolating the formed nano-assembly matrix; and

(iii) introducing the isolated nano-assembly matrix into a cell, a tissue or a living body to regulate or induce physiological conditions or functions that are mediated by the detector materials or the mediator (regulator) materials, thereby diagnosing, preventing or treating the disease related to the physiological conditions or functions.

The present invention also provides a method for diagnosing, preventing or treating disease related to physiological conditions or functions in cells or in vivo, the method comprising the steps of:

(i) providing first mediator (regulator) materials, second mediator (regulator) materials, detector materials and a nano-assembly matrix-forming material to the same field or system;

(ii) forming a nano-assembly matrix by the interaction between the first mediator (regulator) materials, displaying the detector materials at high density by the second mediator (regulator) materials, and

(iii) introducing the isolated nano-assembly matrix into a cell, a tissue or a living body to regulate or induce physiological conditions or functions that are mediated by the detector materials or the mediator (regulator) materials, thereby diagnosing, preventing or treating the disease related to the physiological conditions or functions.

The present invention also provides a method for diagnosing, preventing or treating disease related to physiological conditions or functions in cells or in vivo, the method comprising the steps of:

(i) providing detector materials and a nano-assembly matrix-forming material to the same field or system;

(ii) displaying the detector materials on nano-matrices, and isolating the formed nano-matrices; and

(iii) either introducing the isolated nano-matrices into a cell, a tissue or a living body to regulate or induce physiological conditions or functions that are mediated by the detector materials, or forming a nano-assembly matrix from the isolated nano-matrices in a cell, a tissue or a living body by the interaction between the detector materials or by mediator (regulator) materials interacting with the detector materials, and regulating or inducing physiological conditions or functions that are mediated by the detector materials or mediator (regulator) materials displayed on the nano-assembly matrix at high density, thereby diagnosing, preventing or treating the disease related to physiological conditions or functions in cells or in vivo.

The present invention also provides a method for diagnosing, preventing or treating disease related to physiological conditions or functions in cells or in vivo, the method comprising the steps of:

(i) providing detector materials and a nano-assembly matrix-forming material to the same field or system;

(ii) forming a nano-assembly matrix by the interaction between the detector materials to display the detector materials at high density, and isolating the formed nano-assembly matrix; and

(iii) introducing the isolated nano-assembly matrix into a cell, a tissue or a living body to regulate or induce physiological conditions or functions that are mediated by the detector materials, thereby diagnosing, preventing or treating the disease related to physiological conditions or functions in cells or in vivo.

Herein, regulatory materials capable of interacting with the detector materials displayed on the nano-assembly matrix may additionally be provide and displayed on the nano-assembly matrix, and then the nano-assembly matrix may be isolated, whereby physiological conditions or functions that are mediated by the detector materials or the regulator materials can be regulated or induced, thereby diagnosing, preventing or treating the physiological conditions or functions that are mediated by the detector materials or the regulator materials.

Other features and embodiments of the present invention will be more apparent from the following detailed descriptions and the appended claims.

### BRIEF DESCRIPTION OF THE DRAWINGS

FIG. 1 is a schematic view showing artificially inducing relevant physiological regulation using bioactive materials displayed on a nano-assembly matrix at high density in cells or in vivo. The detector materials that are bioactive materials in FIGS. 2 to 8 correspond to bait materials displayed at high density.

FIG. 2 is a schematic view showing a construct in which X is displayed on a nano-assembly matrix when the nano-assembly matrix is formed by the direct interaction between mediator (regulator) materials A and B or the indict interaction through C. In FIG. 2, A, B, C and X are the same or different materials, and N is a nano-assembly matrix-forming material. Relevant specific physiological regulation is induced by the X, A, B and C materials displayed on N at high density.

FIG. 3 is a schematic view showing a construct in which the formation of a nano-assembly matrix is induced by the interaction between first mediator (regulator) materials A, B and C while X is displayed on the nano-assembly matrix by the interaction between second mediator (regulator) materials D, E and F. In FIG. 3, A, B, C, D, E, F and X are the same or different materials, and N is a nano-assembly matrix-forming material. Relevant specific physiological regulation is induced by the X, A, B, C, D, E and F materials displayed on N at high density.

FIGS. 4 (A) and 4(B) are schematic views showing constructs in which the direct interaction between X and X or X and Y or the indirect interaction through A on a nano-matrix. In FIG. 4, X, Y and A are the same or different materials, and N is a nano-assembly matrix-forming material. Relevant specific physiological regulation is induced by the X, Y and A materials displayed on N at high density.

FIGS. 5(A), 5(B) and 5(C) are schematic views showing constructs in which the direct interaction between X and Y or the indirect interaction through A on a nano-assembly matrix occurs. In FIG. 5, X, Y and A are the same or different materials, and N and N′ are nano-assembly matrix-forming materials. In FIG. 5(A), the same nano-assembly matrix-forming materials are used, and in FIG. 5(B), different nano-assembly matrix-forming materials are used, and in FIG. 5(C), the same detector materials are used. Relevant specific physiological regulation is induced by the X, Y and A materials displayed on N at high density.

FIGS. 6(A), 6(B) and 6(C) are schematic views showing constructs in which the interaction between X and Y on a nano-assembly matrix occurs indirectly through the mediator (regulator) materials A and B or A, B and C fused to the detector materials. In FIG. 6, X, Y, A, B and C are the same or different materials, and N and N′ are nano-assembly matrix-forming materials. In FIG. 6(A), the same nano-assembly matrix-forming materials are used, and in FIG. 6(B), different nano-assembly matrix-forming materials are used, and in FIG. 6(C), the same detector materials are used. Relevant specific physiological regulation is induced by the X, Y, A, B and C materials displayed on N at high density.

FIGS. 7(A) and 7(B) are schematic views showing constructs in which the direct interaction between A and B or the indict interaction through C on a nano-assembly matrix occurs while the interaction between X and Y occurs. In FIG. 7, A, B, C, X and Y are the same or different materials, and N and N′ are nano-assembly matrix-forming materials. In FIG. 7(A), the same nano-assembly matrix-forming materials are used, and in FIG. 7(B), different nano-assembly matrix-forming materials are used. Relevant specific physiological regulation is induced by the X, Y, A, B and C materials displayed on N at high density.

FIGS. 8(A) and 8(B) are schematic views showing constructs in which the direct interaction between A and B or the indict interaction through C on a nano-assembly matrix occurs while the interactions between X and Y and between X and X occur. In FIG. 8, A, B, C, X and Y are the same or different materials, and N is a nano-assembly matrix-forming material. In FIG. 8(A), X and X do not interact with each other, and in FIG. 8(B), X and X interact with each other. Relevant specific physiological regulation is induced by the X, Y, A, B and C materials displayed on N at high density.

FIG. 9 shows the structure of nano-matrices formed by the interaction and self-assembly between the self-association domains of calcium/calmodulin-dependent kinase II (CAM) protein.

FIG. 10 shows the fundamental structure of a fusion protein for making a nano-matrix by fusing a detector protein (fluorescence protein) to the self-association domain of calcium/calmodulin-dependent kinase II (CAM) protein. The detector protein may be fused not only to the N-terminal end of calcium/calmodulin-dependent kinase II (CAM) protein, but also to the C-terminal end. Based on this, calcium/calmodulin-dependent kinase II (CAM) protein is used as a nano-assembly matrix-forming material.

FIG. 11 shows the results of imaging mCerulean (FIG. 11A) and mCitrine (FIG. 11B) in order to examine the interaction between FKBP-mCerulean-FT and FRB-mCitrine-CAM on nano-matrices formed from the self-association domain of calcium/calmodulin-dependent kinase II (CAM) protein and nano-matrices formed from ferritin (FT) protein in HeLa cells, in the presence or absence of a mediator (regulator) material (rapamycin). FIG. 11 shows that the self-association domain of calcium/calmodulin-dependent kinase II (CAM) protein is used as a nano-assembly matrix-forming material according to the scheme of FIG. 5, like ferritin (FT) protein.

FIG. 12 shows the results of imaging mCerulean (FIG. 12A) and mCitrine (FIG. 12B) in order to examine the interaction between FKBP-mCerulean-CAM and FRB-mCitrine on a nano-assembly matrix, formed from the self-association domain of calcium/calmodulin-dependent kinase II (CAM) protein by the first mediator (regulator) material FKBP(F36M)2 in HeLa cells, in the presence or absence of a second mediator (regulator) material (rapamycin). FIG. 12 shows that the self-association domain of calcium/calmodulin-dependent kinase II (CAM) protein is used as a nano-assembly matrix-forming material according to the scheme of FIG. 3, like ferritin (FT) protein.

FIG. 13 shows the results of imaging mCerulean (FIG. 13A) and mCitrine (FIG. 13B) in order to examine the interaction between FKBP-mCherry-FT and FRB-EGFP on a nano-assembly matrix, formed from ferritin (FT) protein by the first mediator (regulator) material FKBP(F36M)2 in HeLa cells, in the presence or absence of a second mediator (regulator) material (rapamycin). FIG. 13 shows that ferritin protein is used as a nano-assembly matrix-forming material according to the scheme of FIG. 3.

FIG. 14 is a graphic diagram showing the regulation of intracellular signaling and transcriptional activity of NFkB by a material (Rel) displayed at high density according to the scheme of FIG. 3 on a nano-assembly matrix formed from the self-association domain of calcium/calmodulin-dependent kinase II (CAM) protein.

FIG. 15 is a graphic diagram showing the regulation of intracellular signaling and transcriptional activity of NFkB by a material (Rel) displayed at high density according to the scheme of FIG. 3 on a nano-assembly matrix formed from ferritin (FT) protein.

FIG. 16 is a graphic diagram showing the regulation of intracellular signaling and transcriptional activity of NFkB by a material (Rel) displayed at high density according to the scheme of FIG. 4 on nano-matrices formed from ferritin (FT) protein.

FIG. 17 is a graphic diagram showing the regulation of intracellular signaling and transcriptional activity of NFkB by a material (Rel) displayed at high density according to the scheme of FIG. 2 on a nano-assembly matrix formed from ferritin (FT) protein.

FIG. 18 is a graphic diagram showing the regulation of intracellular signaling and transcriptional activity of NFkB by a material (Rel) displayed at high density according to the scheme of FIG. 4 on nano-matrices formed from the self-association domain of calcium/calmodulin-dependent kinase II (CAM) protein.

FIG. 19 is a graphic diagram showing the regulation of intracellular signaling and transcriptional activity of NFkB by a material (Rel) displayed at high density according to the scheme of FIG. 4 on a nano-assembly matrix formed from the self-association domain of calcium/calmodulin-dependent kinase II (CAM) protein and ferritin (FT) protein.

FIG. 20 shows an example of the fundamental structure of a fusion protein for isolating and purifying the nano-matrices or nano-assembly matrix formed from the self-association domain of calcium/calmodulin-dependent kinase II (CAM) protein or ferritin (FT) protein. This fusion protein can be easily isolated and purified.

FIG. 21 shows examples of therapeutic or diagnostic materials displayed at high density on the nano-matrices or nano-assembly matrix formed from the self-association domain of calcium/calmodulin-dependent kinase II (CAM) protein or ferritin (FT) protein.

FIG. 22 is a set of images of some (1-30) of the therapeutic and diagnostic proteins of FIG. 21, which were fused to FRB-mCherry, expressed in cells and treated with rapamycin so as to be displayed at high density on a nano-assembly matrix formed from FKBP(F36M)2-fused ferritin (FT).

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods are those well known and commonly employed in the art.

The definition of main terms used in the present invention is as follows.

As used herein, the term “bioactive material or molecule” can be defined as a material which performs regulatory functions, such as promoting or inhibiting the function of the organism's body during the life of organisms. Such a bioactive material can be obtained from natural products such as animals or plants or extracted or purified from the metabolites of microbial, animal and plant cell lines. Moreover, it can also be obtained by chemical synthesis. Examples of the bioactive material include nucleic acids, nucleotides, proteins, peptides, amino acids, saccharides, lipids, vitamins and chemical compounds.

As used herein, the term “detector material” refers to a bioactive material which can be used to detect the interactions with other bioactive materials.

As used herein, the term “regulator material or molecule” refers to a material which is related directly or indirectly to or interacts with a material that mediates an intracellular function to be regulated. According to the intended use, the regulator material may be either a detector material or a material that binds to and interacts with a detector material. In addition, a material that activates, induces, blocks or inhibits an intracellular function to be mediated by a detector material or a mediator (regulator) may also be used as the regulator material.

As used herein, the term “nano-assembly matrix” refers a high-density larger matrix, which is formed by the interaction of nano-matrices having a specific structural framework and can be readily observed. For example, the nano-assembly matrix is a large matrix formed by the interaction of nano-matrices having a specific structural framework, formed by self-assembly of 24 subunits of a protein such as ferritin. In the present invention, the nano-assembly matrix is used in the same sense as an amplified high-signal-intensity dotted image pattern that can be readily observed. In the art to which the present invention pertains, the terms “nanoclusters” and “nanoassemblies” are used in the same sense as the nano-assembly matrix. In an embodiment of the present invention, a high-density matrix of nano-matrices, formed by the interaction between detector materials or between mediator (regulator) materials, is referred to as the formation of the nano-assembly matrix. The formation of the nano-assembly matrix can be confirmed by the transfer and change of energy signals. In addition, it can also be confirmed by a change in a dotted image pattern having a high signal intensity.

As used herein, the expression “nano-assembly matrix-forming material” refers to any material having the property and function of forming the nano-assembly matrix. For example, the term means a poly/multi-valent material such as ferritin, which has a plurality of the same or different binding moieties and can form an assembly by interaction or self-assembly. In the present invention, the term means a material that forms either an observable high-signal-intensity dotted image pattern by the interaction between the mediator (regulator) molecules or a nano-assembly matrix (i.e., observable high-signal-intensity dotted image pattern) by the interaction between the detector materials.

As used herein, the term “nano-matrices” refers to matrices on which the nano-assembly matrix is based and which are formed by self-assembly of protein or the like. For example, 24 ferritin protein subunits are self-assembled to form a nano-matrix. Particularly, as first demonstrated herein, proteins having a self-association domain, like calcium/calmodulin-dependent kinase II protein, can very easily form nano-matrices, and furthermore, nano-assembly matrices. Thus, it can be seen that materials capable of forming the nano (assembly) matrix according to the present invention include bioactive materials having a self-association domain.

As used herein, the term “mediator (regulator) material” refers to a material inducing the formation of the nano-assembly matrix. This is meant to include all materials capable of inducing the formation of the nano-assembly matrix through direct or indirect binding, interaction or fusion with the nano-assembly matrix-forming material. A material that mediates or regulates the activity of the mediator (regulator) material may also be defined as a mediator (regulator) material in a broad sense. The mediator (regulator) molecules include not only specific compounds or proteins, which induce the formation of the nano-assembly matrix, but also phenomena such as specific mutations, and specific physiological signals. For example, the formation of the nano-assembly matrix can be induced through the interactions between proteins resulting from physiological signals, the interactions between RNA and protein, the use of a specific mutation of a specific protein, or the use of a protein interacting only with a specific compound, and such phenomena and signals are referred to as “mediator (regulator) materials” in the specification of the present invention.

As used herein, the term “display” is meant to include exposing a material directly to the inside or the outside of a nano-matrix or a nano-assembly matrix, or exposing the material indirectly through another material, or loading a material into a nano-matrix or a nano-assembly matrix. Physiological activities or functions can be regulated or induced by the loaded material that is exposed due to the dis-assembly of the nano-matrix or the nano-assembly matrix.

As used herein, the term “physiological regulation” is meant to include the modification and regulation of various physiological functions or conditions that are regulated or induced in cells or in vivo. Thus, the term also includes the regulation or induction of all the physiological functions or conditions related to diseases.

Hereinafter, the present invention will be described in detail.

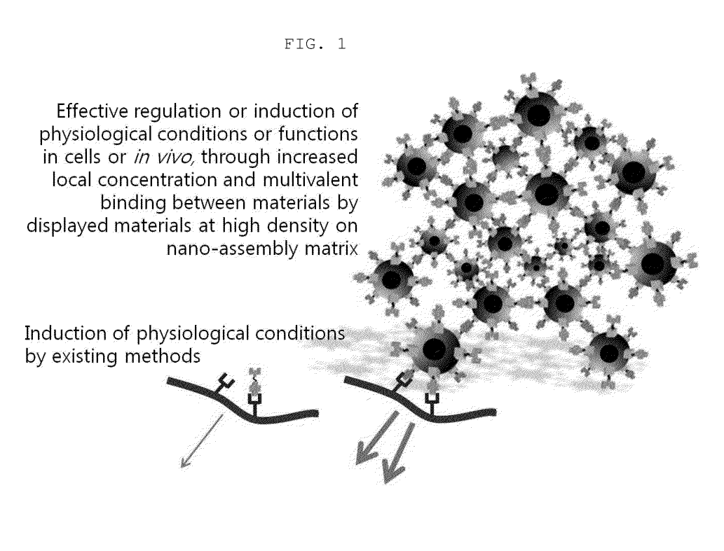

The present invention is directed to a method in which bioactive materials that interact with each other are displayed on a nano-assembly matrix or a nano-matrix at high density to inhibit or activate physiological conditions or functions, which are mediated by the displayed materials or materials interacting therewith, thereby regulating or inducing various physiological conditions or functions in cells or in vivo (FIG. 1). This method according to the present invention can be embodied by the following methods of first to fifth embodiments.

The first and second embodiments of the present invention are methods of regulating or inducing physiological conditions or functions in cells or in vivo using the interaction between materials. In these methods, a nano-assembly matrix is formed by mediator (regulator) materials, and then physiological conditions or functions that are mediated by the detector materials or mediator (regulator) materials displayed on the formed nano-assembly matrix at high density can be regulated or induced.

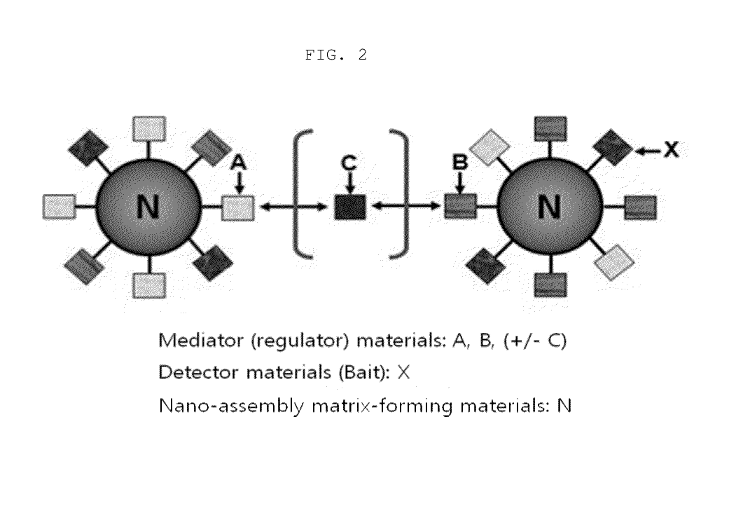

Specifically, the first embodiment of the present invention is a method for regulating or inducing physiological conditions or functions in cells or in vivo (FIG. 2), the method comprising the steps:

(i) providing mediator (regulator) materials, detector materials and a nano-assembly matrix-forming material to the same field or system; and

(ii) forming a nano-assembly matrix by the interaction between the mediator (regulator) materials to display the detector materials at high density, thereby regulating or inducing physiological conditions or functions that are mediated by the mediator (regulator) materials or the detector materials.

A modification of the first embodiment may provide a method for screening a material that regulates or induces physiological conditions or functions in cells or in vivo, the method comprising the steps of:

(i) providing mediator (regulator) materials, detector materials and a nano-assembly matrix-forming material to the same field or system;

(ii) forming a nano-assembly matrix by the interaction between the mediator (regulator) materials to display the detector materials at high density;

(iii) providing target candidates to the nano-assembly matrix; and

(iv) selecting, as the material that regulates or induces physiological conditions or functions in cells or in vivo, a target candidate corresponding to a case in which physiological conditions or functions in the presence of the target candidate change compared to physiological conditions or functions in the absence of the target candidate.

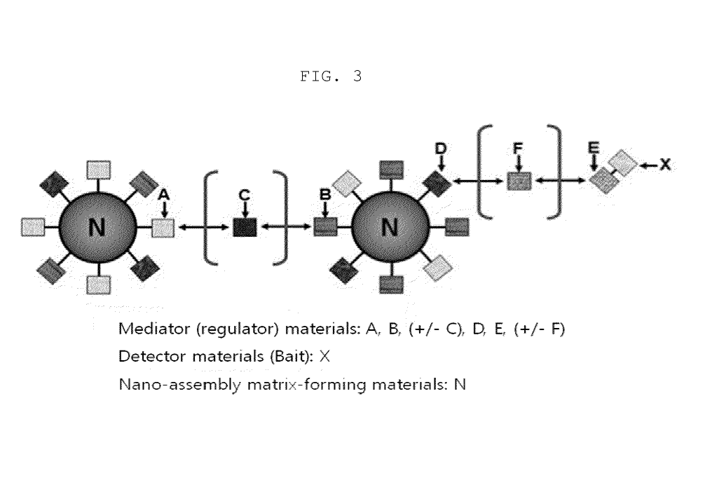

The second embodiment of the present invention is a method for regulating or inducing physiological conditions or functions in cells or in vivo (FIG. 3), the method comprising the steps of:

(i) providing first mediator (regulator) materials, second mediator (regulator) materials, detector materials and a nano-assembly matrix-forming material to the same field or system;

(ii) forming a nano-assembly matrix by the interaction between the first mediator (regulator) materials, and displaying the detector materials at high density by the interaction between the second mediator (regulator) materials, thereby regulating or inducing physiological conditions or functions, which are mediated by the mediator (regulator) materials or the detector materials.

A modification of the second embodiment may provide a method for screening a material that regulates or induces physiological conditions or functions in cells or in vivo, the method comprising the steps of:

(i) providing first mediator (regulator) materials, second mediator (regulator) materials, detector materials and a nano-assembly matrix-forming material to the same field or system;

(ii) forming a nano-assembly matrix by the interaction between the first mediator (regulator) materials, and displaying the detector materials at high density by the interaction between the second mediator (regulator) materials;

(iii) providing target candidates to the nano-assembly matrix; and

(iv) selecting, as the material that regulates or induces physiological conditions or functions in cells or in vivo, a target candidate corresponding to a case in which physiological conditions or functions in the presence of the target candidate change compared to physiological conditions or functions in the absence of the target candidate.

In step (i) of each of the methods according to the first and second embodiments, a material that mediates or regulates the interaction between the mediator (regulator) materials may be added.

The methods according to the first and second embodiments commonly comprise forming a nano-assembly matrix is formed by the interaction between mediator (regulator) materials that are known to interact with each other, and then regulating physiological functions that are regulated by the mediator (regulator) materials or the detector materials displayed on the formed nano-assembly matrix at high density.

However, the biggest difference between the first method and the second method is that, in the step of forming the nano-assembly matrix by the interaction between the mediator (regulator) materials in the first method, the detector materials bound to the nano-assembly matrix-forming material participate in forming the nano-assembly matrix by the interaction between the mediator (regulator) materials, whereas, in the step of the second method, the nano-assembly matrix is formed only by the nano-assembly matrix-forming material and the first mediator (regulator) materials, and then second mediator (regulator) materials are bound to the resulting nano-assembly matrix. Herein, an additional regulator material that mediates or regulates the interaction between the mediator (regulator) materials (including the first mediator (regulator) materials or the second (regulator) materials) may further be used. As used herein, the term “additional regulator material” refers to a bioactive material capable of binding to the regulator materials displayed on the previously formed nano-assembly matrix, and the kind or number of additional materials is not limited, as long as they can interact with the regulator materials displayed on the nano-assembly matrix. In other words, one or more additional regulator materials capable of interacting with the displayed regulator materials may be used.

FIG. 2 schematically shows the first method of the present invention.

FIG. 3 schematically shows the second method of the present invention.

An example of the present invention will be described with reference to a schematic view of FIG. 3. As shown in FIG. 3, on calcium/calmodulin-dependent kinase II (CAM) protein or ferritin protein (corresponding to N in FIG. 3) that is a nano-assembly matrix-forming material, the mediator (regulator) materials FKBP(F36M)2 (corresponding to A, B and D in FIG. 3) caused by a specific mutation in FKBP protein present as a monomer spreading in cells are self-associated to induce the spontaneous formation of a nano-assembly matrix. Then, the resulting material and the second mediator (regulator) materials FRB (E in FIG. 3) and RelA (X in FIG. 3) are recruited and displayed on the nano-assembly matrix by the material rapamycin (F in FIG. 3) that regulates the interaction between the second mediator (regulator) materials. As can also be seen from the results of the example, FKBP(36M)2 and FRB are displayed on the surface of the nano-assembly matrix by treatment with rapamycin. In addition, when treatment with TNF-a (Y) as a prey material for the detector material RelA is performed, the prey material such as TNF-a can induce a specific reaction (change in NFkB activity) by binding to the detector material (RelA) that is the partner thereof (see the example of the present invention).

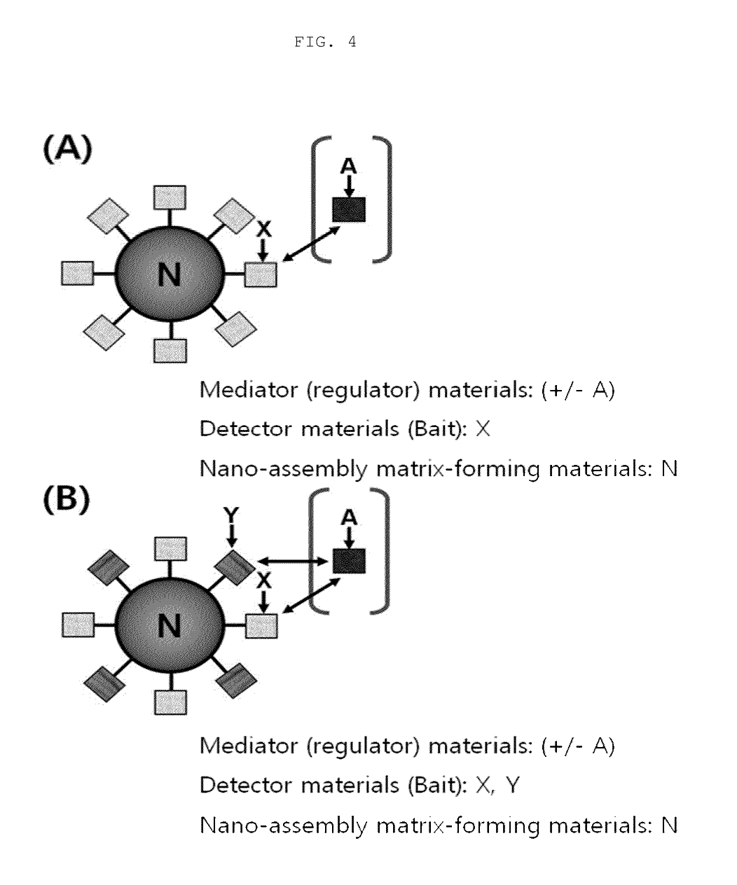

The third embodiment of the present invention is a method for regulating or inducing physiological conditions or functions in cells or in vivo (FIG. 4), the method comprising the steps of:

(i) providing detector materials and a nano-assembly matrix-forming material to the same field or system; and

(ii) displaying the detector materials on nano-matrices at high density, thereby regulating or inducing physiological conditions or functions that are mediated by the detector materials.

A modification of the third embodiment may provide a method for screening a material that regulating or inducing physiological conditions or functions in cells or in vivo, the method comprising the steps of:

(i) providing detector materials and a nano-assembly matrix-forming material to the same field or system; and

(ii) displaying the detector materials on nano-matrices at high density;

(iii) either providing target candidates to the nano-matrices, or isolating the nano-matrices, introducing the isolated nano-matrices into a cell, a tissue or a living body, and providing target candidates to a nano-assembly matrix formed by the interaction between the detector materials or by added mediator (regulator) materials; and

(iv) selecting, as the material that regulating or inducing physiological conditions or functions in cells or in vivo, a target candidate corresponding to a case in which physiological conditions or functions in the presence of the target candidate change compared to physiological conditions or functions in the absence of the target candidate change.

Another modification of the third embodiment of the present invention may provide a method for screening a material that regulates or induces physiological conditions or functions in cells or in vivo, the method comprising the steps of:

(i) providing first mediator (regulator) materials, detector materials and a nano-assembly matrix-forming material to the same field or system;

(ii) displaying the first mediator (regulator) materials and the detector materials on nano-matrices at high density;

(iii) either providing target candidates to the nano-matrices, or isolating the nano-matrices, introducing the isolated the nano-matrices into a cell, a tissue or a living body, and providing target candidates to a nano-assembly matrix formed by the interaction between the first (mediator) materials or by added second mediator (regulator) materials; and

(iv) selecting, as the material that regulating or inducing physiological conditions or functions in cells or in vivo, a target candidate corresponding to a case in which physiological conditions or functions in the presence of the target candidate change compared to physiological conditions or functions in the absence of the target candidate change.

In the third method, the nano-assembly matrix-forming material forms nano-matrices by self-assembly or the interaction or self-assembly between a plurality of interacting sites thereof, whereby the detector materials bound to the nano-assembly matrix-forming material are displayed on the nano-matrices at high density, thereby regulating or inducing physiological conditions or functions that are mediated by the detector materials.

Herein, the detector materials that are bound to the nano-assembly matrix-forming material may be two or more different detector materials and may further include a material that regulates the interactions between the two or more different detector materials (FIG. 4B).

Herein, an additional regulator material that mediates or regulates the interaction between the mediator (regulator) materials (including the first mediator (regulator) materials or the second (regulator) materials) may further be used. As used herein, the term “additional regulator material” refers to a bioactive material capable of binding to the regulator materials displayed on the previously formed nano-assembly matrix, and the kind or number of additional materials is not limited, as long as they can interact with the regulator materials displayed on the nano-assembly matrix. In other words, one or more additional regulator materials capable of interacting with the displayed regulator materials may be used.

FIG. 4 schematically shows the third method of the present invention.

An embodiment of the present invention will be described with reference to the schematic view of FIG. 4. As shown in FIG. 4, the detector material Rel (corresponding to X in FIG. 4) was displayed at high density on a nano-matrix formed by fusing Rel to ferritin protein (corresponding to N in FIG. 4) that is a nano-assembly matrix forming material, and whether a specific intracellular reaction (change in NFkB activity) can be TNF-a interacting with the displayed Rel was examined (see the example of the present invention).

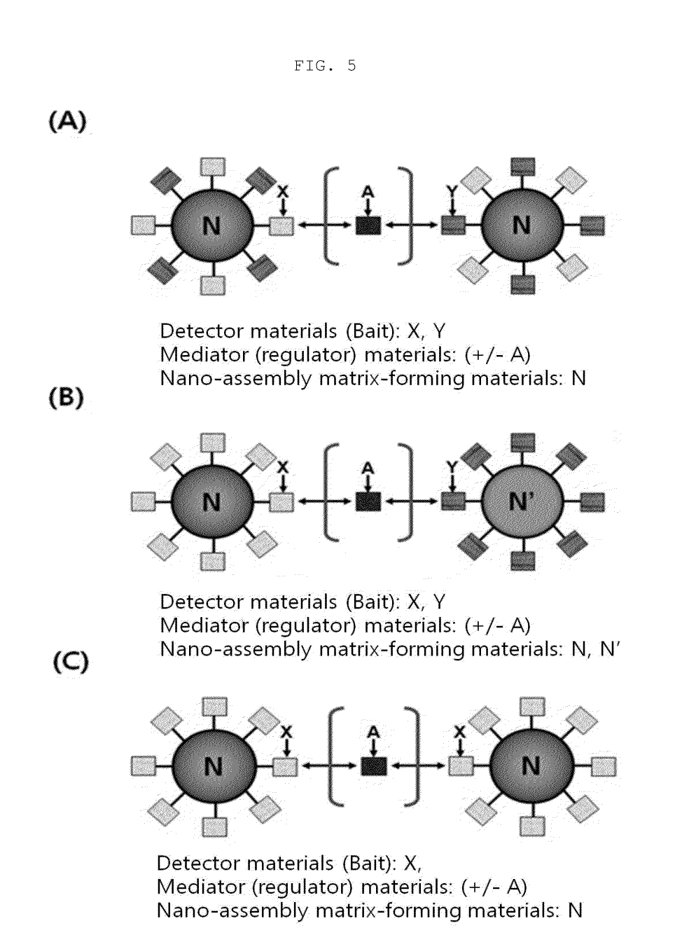

The fourth embodiment of the present invention is directed to a method for regulating or inducing physiological conditions or functions in cells or in vivo (FIG. 5), the method comprising the steps of:

(i) providing detector materials and nano-assembly matrix-forming materials to the same field or system; and

(ii) forming a nano-assembly matrix by the interaction between the detector materials to display the detector materials at high density, thereby regulating or inducing physiological conditions or functions that are mediated by the detector materials.

A modification of the fourth embodiment of the present invention may provide a method for screening a material that regulates or induces physiological conditions or functions in the cells or in vivo, the method comprising the steps of:

(i) providing detector materials and nano-assembly matrix-forming materials to the same field or system;

(ii) forming a nano-assembly matrix by the interaction between the detector materials to display the detector materials at high density;

(iii) providing target candidates to the nano-assembly matrix; and

(iv) selecting, as the material that regulating or inducing physiological conditions or functions in cells or in vivo, a target candidate corresponding to a case in which physiological conditions or functions in the presence of the target candidate change compared to physiological conditions or functions in the absence of the target candidate change.

In the fourth method, the nano-assembly matrix is formed by the direct or indirect interaction between the detector materials to display the detector materials on the formed nano-assembly matrix at high density, thereby physiological conditions or functions that are mediated by the detector materials. Herein, the detector materials that interact with each other so as to form the nano-assembly matrix may be the same or different materials. In addition, the nano-assembly matrix-forming materials may be the same (FIG. 5A) or different (FIG. 5B).

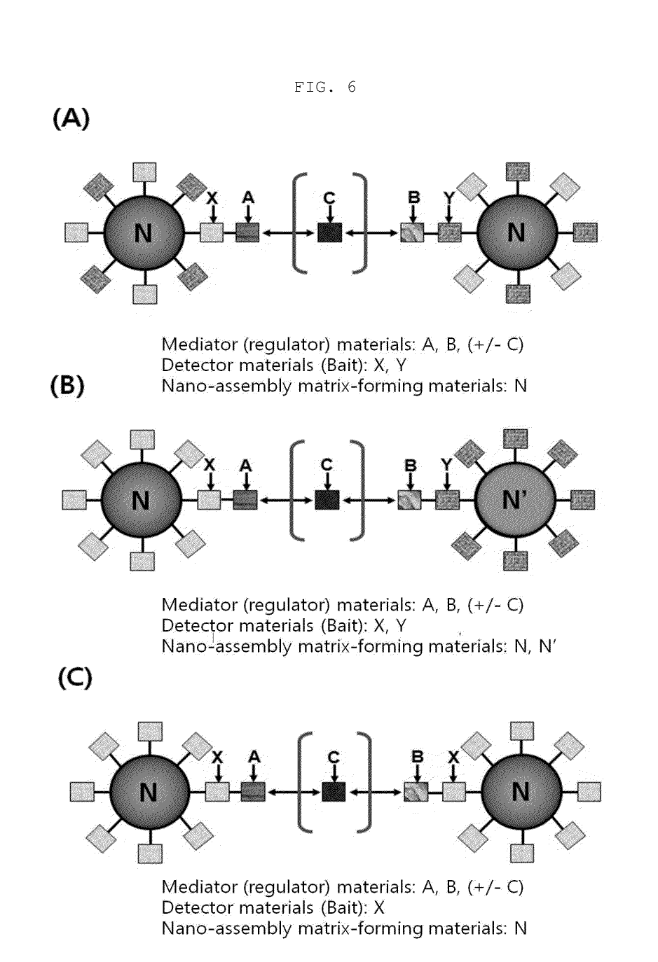

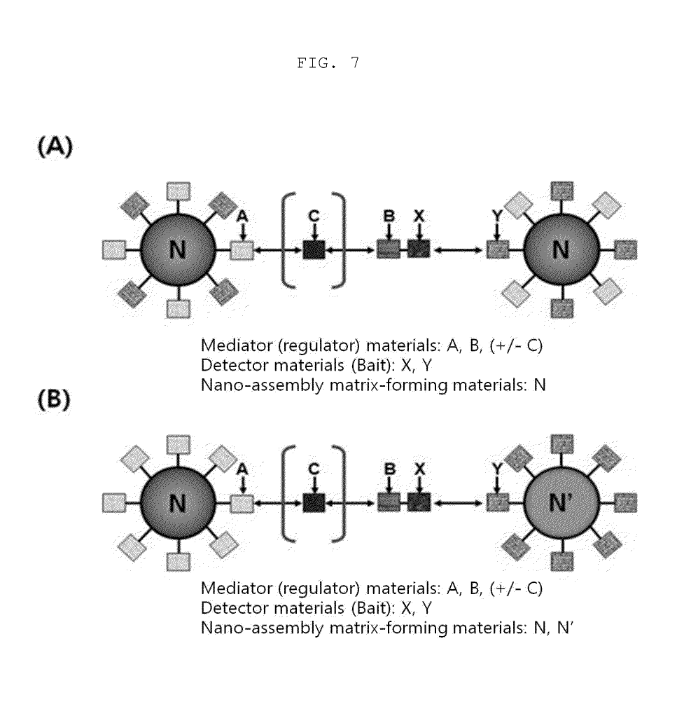

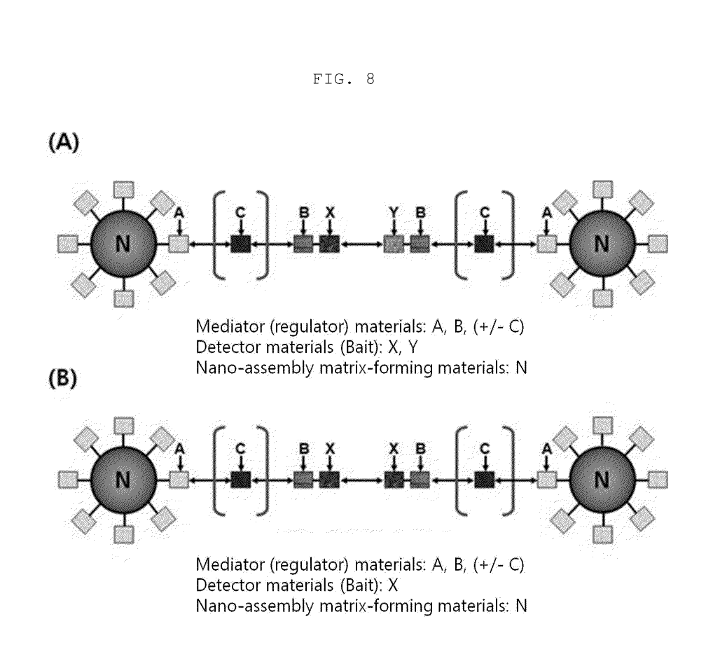

In addition, one or more mediator (regulator) materials that mediate (regulate) the interaction between the detector materials or between the detector materials and the nano-assembly matrix-forming materials may additionally be added. Herein, the mediator (regulator) materials may be added with the mediator (regulator) materials fused to the detector materials(FIGS. 6 to 8).

In the fifth method, the detector materials may be a bait and a prey, known to interact with each other, or a bait and a prey, known to interact with each other as described in Korean Patent Application No. 10-2008-0079957 filed in the name of the present inventors. When regulator materials capable of binding to the detector materials are displayed on the nano-assembly matrix formed by the interaction between the detector materials, physiological functions that are mediated by the detector materials or the regulator materials can be inhibited or activated.

FIG. 5 schematically shows the fourth method of the present invention. As shown in FIG. 5, the detector materials FRB and FKBP (corresponding to X in FIG. 5) were fused to calcium/calmodulin-dependent kinase II (CAM) protein and ferritin protein (corresponding to N in FIG. 5) that are nano-assembly matrix-forming materials, and the resulting material was treated with a rapamycin analogue as a mediator (regulator) material (corresponding A in FIG. 5) to form a nano-assembly matrix having the FRB, FKBP and Rel displayed thereon. Then, whether a specific intracellular reaction (change in NFkB activity) can be TNF-a interacting with the displayed Rel was examined (see the example of the present invention).

In the methods according to the present invention, regulator materials capable of interacting with the displayed detector materials may additionally be provided and displayed at high density, thereby regulating or inducing physiological conditions or functions that are mediated by the detector materials or the regulator materials.

In the above-described methods according to the present invention, the binding between the materials used in the present invention, including nano-assembly matrix-forming materials, bait molecules, prey molecules, mediator (regulator) molecules inducing nano-assembly matrix formation and labels, may include physical, chemical, electrostatic or biological direct or indirect binding. Among them, when biological binding occurs, a probe comprising an antibody, a protein, a protein domain, a motif, a peptide or the like may be used.

The detector materials or mediator (regulator materials) displayed at high density on the nano-assembly matrices formed by the methods of the present invention can trap or sequester other materials in cells or in vivo, which interact therewith. Thus, it is possible to regulate or induce physiological conditions or functions that are mediated by the other materials trapped or sequestered by the bait materials, prey materials or regulator materials displayed in the nano-assembly matrices.

In the methods of the present invention, any detector materials may be bound directly or indirectly to nano-assembly matrix-forming materials, while prey materials may be bound to the nano-assembly matrix-forming materials. In addition, mediator (regulator) materials may be bound to nano-assembly matrix-forming materials, while mediator (regulator) materials may be bound to bait materials and prey materials.

The regulator material may be a material that is involved in the on/off of changes in physiological functions (inhibition or activation of physiological functions) of interest. As described above, the assembly or dis-assembly of nano-matrices or the display of specific materials in the nano-assembly matrix can be regulated using bioactive molecules that interact with each other, thereby regulating physiological functions in an intracellular or in vivo environment.

In addition, by examining a change in physiological function of interest, for example, the stimulation of activity of a specific material, or an increase or decrease in the production of a specific material, whether the physiological function of cells was regulated can be determined by the above-described method.

In the method of the present invention, the nano-assembly matrix-forming molecule, the detector material or the mediator (regulator) material may be can be labeled with a label. Herein, examples of the label include, but are not limited to, magnetic materials, radioactive materials, enzymatic materials for ELISA, fluorescent materials, and luminescent materials. Examples of the fluorescent materials include fluorescent dyes, fluorescent proteins and fluorescent nanoparticles.

In addition, in the method of the present invention, the bait molecule, the prey material or the mediator (regulator) material may be a bioactive molecule. Herein, the bioactive molecule may be one or more selected from the group consisting of nucleic acids, nucleotides, proteins, peptides, amino acids, saccharides, lipids, vitamins, and chemical compounds, but is not limited thereto.

The method of the present invention can be performed in vitro or in vivo. When the method of the present invention is performed in vivo, it can be performed in eukaryotic cells, prokaryotic cells, the living tissue and cells of mammals including humans, or the living tissue and cells of plants. Particularly, the method of the present invention can be performed in the living cells or tissues of Zebra fish, C. elegans, yeast, flies or frogs.

The nano-assembly matrix-forming material, the bait material, the prey material, the mediator material (material inducing nano-assembly matrix formation) and the label, which are used in the present invention, can be easily introduced into cells using widely known general methods. For example, introduction of these materials into cells can be performed by any one method selected from the group consisting of direct injection, a method employing a transducible peptide, a fusogenic peptide, a lipid delivery system or a combination thereof, electroporation, magnetofection, and parenteral administration, oral administration, intranasal administration, subcutaneous administration, aerosolized administration and intravenous administration into mammals including humans.

In the methods of the present invention, formation of the nano-assembly matrix can be examined using a label. Particularly, the detector molecule, the nano-assembly matrix-forming material or the mediator (regulator) molecule, which are used in the present invention, may be labeled with a label. If necessary, a radioactive label, a fluorescent material or a luminescent material may be used as a label on the nano-assembly matrix formed by the interaction between specific molecules according to the present invention. Examples of the radioactive label that may be used in the present invention include all known labels, including ³²P, ³⁵S, ³H and ¹⁴C. Moreover, fluorescent materials that may be used as labels in the present invention show fluorescence by themselves or by interaction with other materials and include, for example, fluorescent dyes such as FITC, rhodamine and the like; fluorescent proteins such as ECFP, TagCFP, mTFP1, GFP, YFP, CFP and RFP; tetracysteine motifs; and fluorescent nanoparticles. In addition, luminescent materials that may be used as labels in the present invention shows luminescence by themselves or by interaction with other materials and include, for example, luciferase and the like.

In the methods of the present invention, formation of the nano-assembly matrix can be measured or detected using widely known general methods, including a magnetic method, a radioactive method, a method using an enzyme for ELISA, a method of detecting a fluorescent or luminescent material, an optical method, or a method employing a microscope, an imaging system, a scanner, a reader, a spectrophotometer, MRI (magnetic resonance imaging), SQUID, an MR relaxometer, FACS (fluorescene associated cell sorting), a fluorometer or a luminometer. In addition, the nano-assembly matrix can be isolated using these methods.

Additionally, regulator molecules can be loaded at high density either into the nano-assembly matrix formed by the method of the present invention or into the nano-matrices, and physiological activities or functions can be regulated or induced by the loaded molecules that are exposed as a result of the dis-assembly of the nano-assembly matrix or the nano-matrices.

Hereinafter, the components that are used in the methods of the present invention will be described in detail.

The “nano-assembly matrix-forming materials” are poly/multi-valent materials having a plurality of the same or different binding moieties and can form matrices by the interaction or self-assembly between them. Preferably, materials that can form matrices by self-assembly are used. These matrices preferably consist of nano-sized particles

Preferred examples of materials that form nano-assembly matrices by self-assembly may include proteins having self-assembly or self-association domains, for example, ferritin, ferritin-like protein, DPS (DNA binding protein from starved cells), DPS-like protein, HSP (heat shock protein), magnetosome protein, viral protein, calcium/calmodulin-dependent kinase II, and dsRed. Moreover, a variety of chemically synthesized nanoparticles can also form nano-assembly matrices. For example, various kinds of nanoparticles, including gold nanoparticles, Q dots or magnetic nanoparticles, may be used. In one example of the present invention, among molecules or proteins that can form nano-matrices (nano-sized unit matrices) by self-assembly, the ferritin protein was used.

The ferritin protein forms a spherical nanoparticle matrix by self-assembly of 24 ferritin subunits, has an outer diameter of about 12 nm and an inner diameter of about 9 nm and contains more than 2500 iron atoms (Chasteen, N. D. Struc. Biol. 126:182-194, 1999). If a nano-assembly matrix is formed by the interaction between the detector molecules or between the between the mediator (regulator) molecules, which occurs on the surface of the nanoparticle matrix formed by the ferritin protein, the interaction can be dynamically detected by analyzing a label (such as a fluorescent, luminescent, magnetic or radioactive material) bound to the detector molecules or the mediator (regulator) molecules, using an analytical device such as a microscope.

The detector molecules that are used in the present invention may be any bioactive molecules that interact with each other. The bioactive molecules are materials that show physiological activity in vivo and can interact with various bioactive molecules in the human body to regulate the function or activity thereof. Preferred examples of the bioactive molecules include nucleic acids, mono-/oligo-/poly-nucleotides, proteins, mono-/oligo-/poly-peptides, amino acids, mono-/oligo-/poly-saccharides, lipids, vitamins, chemical compounds, and small molecules constituting these materials.

Specific examples of the interaction between the bait and the prey may include the interaction between drug-drug targets, the interaction between FRB and FKBP, which are the pharmaceutically relevant binding partners of a Rapamycin compound, the interaction between an FK506 compound and an FKBP protein which is the pharmaceutically relevant partner thereof, the interaction between an AP1510 compound and an FKBP protein, the interaction between an IkBα protein and an RelA which is the binding partner thereof, the interaction between an IkBα protein, which is regulated according to a physiological signal of TNFa, and a bTrCP or IKKb protein which is the binding partner of the IkBα protein, the intracellular interaction (let-7b miRNA binding to lin-28 mRNA) of miRNA with mRNA, the interaction of an Ago2 proteion with miRNA, the interaction of an MS2 protein with an MS2-binding mRNA site, the intracellular interaction of a DHFR protein with an MTX compound, etc.

The mediator (regulator) materials which regulate the interaction between detection materials are materials that activate the interaction between the detection materials to mediate (regulate) the binding between the detection materials, and as the mediator (regulator) materials, any bioactive molecules or compounds may be used without limitation, as long as they exhibit the above function. However, molecules interacting specifically with the detection materials pair are preferably used. Because the nano-assembly matrix is formed by the interaction between the detection materials, the materials that mediate the interaction between the detection materials are considered to fall within the scope of the mediator (regulator) materials which induce the formation of nano-assembly matrices as defined in the present invention.

To mediate (regulate) the interaction between the detection materials, a protein which is regulated by an external signal may be used. Alternatively, the property of miRNA binding specifically to its target mRNA may also be used. In an example of the present invention, when a FKBP-FRB pair was used, rapamycin was used as a mediator (regulator) material.

The mediator (regulator) materials which induce the formation of nano-assembly matrices in the present invention are meant to include all materials which can interact directly or indirectly with each other on the surface of the nano-assembly matrix-forming materials to form nano-assembly matrices. Such materials that mediate or regulate the activity of the mediator (regulator) materials are also considered as mediator (regulator) materials in a broad sense.

As such mediator (regulator) materials, any materials may be used without limitation, as long as they exhibit the function of inducing the formation of nano-assembly matrices. Accordingly, all the materials or phenomena that can induce the formation of nano-assembly matrices by specific phenomena, such as either the binding between materials interacting specifically with each other or mutations can be understood as mediator (regulator) materials. Namely, the term “mediator (regulator) materials” as used herein is meant to include all specific materials, specific phenomena or specific interactions. Such mediator (regulator) materials may be used in a combination of two or more thereof.

The nano-assembly matrices or nano-matrices isolated by the methods of the present invention can be used as compositions vaccination, prevention, material delivery or treatment against diseases related to physiological conditions or functions in cells or in vivo either by the detector materials or mediator (regulator) materials displayed thereon at high density or by materials such as drugs, which are trapped by the detector or mediator (regulator) materials or loaded into the nano-assembly matrices or nano-matrices.

Therefore, in a first embodiment of another aspect of the present invention, there is provided a composition for vaccination, prevention, material delivery or treatment against disease related to physiological conditions or functions in cells or in vivo, the composition comprising a nano-assembly matrix isolated by a method comprising the steps of:

(i) providing mediator (regulator) materials, detector materials and a nano-assembly matrix-forming material to the same field or system; and

(ii) forming a nano-assembly matrix by the interaction between the mediator (regulator) materials to display the detector materials at high density, and isolating the formed nano-assembly matrix.

In a second embodiment of the present invention, there is provided a composition for vaccination, prevention, material delivery or treatment against disease related to physiological conditions or functions in cells or in vivo, the composition comprising a nano-assembly matrix isolated by a method comprising the steps of:

(i) providing first mediator (regulator) materials, second mediator (regulator) materials, detector materials and a nano-assembly matrix-forming material to the same field or system; and

(ii) forming a nano-assembly matrix by the interaction between the first mediator (regulator) materials, displaying the detector materials at high density by the interaction between the second mediator (regulator) materials, and isolating the formed nano-assembly matrix.

In a third embodiment of the present invention, there is provided a composition for vaccination, prevention, material delivery or treatment against disease related to physiological conditions or functions in cells or in vivo, the composition comprising a nano-assembly matrix isolated by a method comprising the steps of:

(i) providing detector materials and a nano-assembly matrix-forming material to the same field or system; and

(ii) displaying the detector materials on nano-matrices at high density, and isolating the formed nano-matrices.

In a modification of the third embodiment of the present invention, there is provided a composition for vaccination, prevention, material delivery or treatment against disease related to physiological conditions or functions in cells or in vivo, the composition comprising a nano-assembly matrix isolated by a method comprising the steps of:

(i) providing mediator (regulator) materials, detector materials and a nano-assembly matrix to the same field or system; and

(ii) displaying the mediator materials and the detector materials on nano-matrices at high density, and isolating the formed nano-matrices.

In another modification of the third embodiment of the present invention, there is provided a composition for vaccination, prevention, material delivery or treatment against disease related to physiological conditions or functions in cells or in vivo, the composition comprising a nano-assembly matrix isolated by a method comprising the steps of:

(i) providing mediator (regulator) materials and a nano-assembly matrix-forming material to the same field or system; and

(ii) displaying the mediator (regulator) materials on nano-matrices, and isolating the formed nano-matrices.

In a fourth embodiment of the present invention, there is provided a composition for vaccination, prevention, material delivery or treatment against disease related to physiological conditions or functions in cells or in vivo, the composition comprising a nano-assembly matrix isolated by a method comprising the steps of:

(i) providing detector materials and a nano-assembly matrix-forming material to the same field or system; and

(ii) forming a nano-assembly matrix by the interaction between the detector materials to display the detector materials at high density, and isolating the formed nano-assembly matrix.

Herein, regulator materials capable of interacting with the displayed detector materials may further be provided and displayed at high density, followed by isolation. In addition, the regulator materials may be loaded into the nano-assembly matrix or the nano-matrices at high density.

Therefore, the present invention provides a method for diagnosing, preventing or treating disease related to physiological conditions or functions in cells or in vivo, the method comprising the steps of:

(i) providing mediator (regulator) materials, detector materials and a nano-assembly matrix-forming material to the same field or system;

(ii) forming a nano-assembly matrix by the interaction between the mediator (regulator) materials to display the detector materials at high density, and isolating the formed nano-assembly matrix; and

(iii) introducing the isolated nano-assembly matrix into a cell, a tissue or a living body to regulate or induce physiological conditions or functions that are mediated by the detector materials or the mediator (regulator) materials, thereby diagnosing, preventing or treating the disease related to the physiological conditions or functions.

The present invention also provides a method for diagnosing, preventing or treating disease related to physiological conditions or functions in cells or in vivo, the method comprising the steps of:

(i) providing first mediator (regulator) materials, second mediator (regulator) materials, detector materials and a nano-assembly matrix-forming material to the same field or system;

(ii) forming a nano-assembly matrix by the interaction between the first mediator (regulator) materials, displaying the detector materials at high density by the second mediator (regulator) materials, and

(iii) introducing the isolated nano-assembly matrix into a cell, a tissue or a living body to regulate or induce physiological conditions or functions that are mediated by the detector materials or the mediator (regulator) materials, thereby diagnosing, preventing or treating the disease related to the physiological conditions or functions.

The present invention also provides a method for diagnosing, preventing or treating disease related to physiological conditions or functions in cells or in vivo, the method comprising the steps of:

(i) providing detector materials and a nano-assembly matrix-forming material to the same field or system;

(ii) displaying the detector materials on nano-matrices, and isolating the formed nano-matrices; and

(iii) either introducing the isolated nano-matrices into a cell, a tissue or a living body to regulate or induce physiological conditions or functions that are mediated by the detector materials, or forming a nano-assembly matrix from the isolated nano-matrices in a cell, a tissue or a living body by the interaction between the detector materials or by mediator (regulator) materials interacting with the detector materials, and regulating or inducing physiological conditions or functions that are mediated by the detector materials or mediator (regulator) materials displayed on the nano-assembly matrix at high density, thereby diagnosing, preventing or treating the disease related to physiological conditions or functions in cells or in vivo.

The present invention also provides a method for diagnosing, preventing or treating disease related to physiological conditions or functions in cells or in vivo, the method comprising the steps of:

(i) providing detector materials and a nano-assembly matrix-forming material to the same field or system;

(ii) forming a nano-assembly matrix by the interaction between the detector materials to display the detector materials at high density, and isolating the formed nano-assembly matrix; and

(iii) introducing the isolated nano-assembly matrix into a cell, a tissue or a living body to regulate or induce physiological conditions or functions that are mediated by the detector materials, thereby diagnosing, preventing or treating the disease related to physiological conditions or functions in cells or in vivo.

Herein, regulatory materials capable of interacting with the detector materials displayed on the nano-assembly matrix may additionally be provide and displayed on the nano-assembly matrix, and then the nano-assembly matrix may be isolated, whereby physiological conditions or functions that are mediated by the detector materials or the regulator materials can be regulated or induced, thereby diagnosing, preventing or treating the physiological conditions or functions that are mediated by the detector materials or the regulator materials.

The present invention also provides the use of the nano-assembly matrix or nano-matrices, isolated by the above-described methods, for preventing or treating disease related to physiological conditions or functions in cells or in vivo.

The above-described pharmaceutical composition for diagnosing, preventing or treating disease may comprise the isolated nano-assembly matrix or nano matrix alone or together with at least one pharmaceutically acceptable carrier, excipient or diluent. The matrix may be contained in the pharmaceutical composition in a pharmaceutically effective amount according to a disease and the severity thereof, the patient's age, weight, health condition and sex, the route of administration, and the period of treatment.

As used herein, the term “pharmaceutically acceptable composition” refers to a composition that is physiologically acceptable and does not cause gastric disorder, allergic reactions such as gastrointestinal disorder or vertigo, or similar reactions, when administered to humans. Examples of said carrier, excipient or diluent may include lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methylhydroxybenzoate, propylhydroxybenzoate, magnesium stearate and mineral oils.

The pharmaceutical composition of the present invention may additionally contain fillers, anti-aggregating agents, lubricants, wetting agents, perfumes, emulsifiers and preservatives. Also, the pharmaceutical composition of the present invention may be formulated using a method well known in the art, such that it can provide the rapid, sustained or delayed release of the active ingredient after administration to mammals. The pharmaceutical composition of the present invention may be in the form of sterile injection solutions, and the like.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Analysis of Formation of Nano-Assembly Matrix and Display of Materials by Ferritin Protein and Self-Association Domain of Calcium/Calmodulin-Dependent Kinase II (CAM) Protein

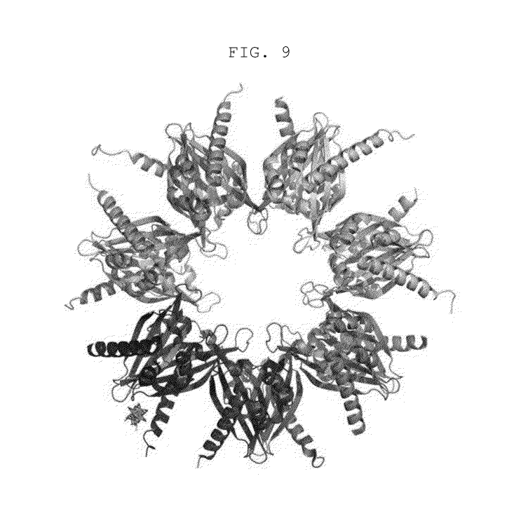

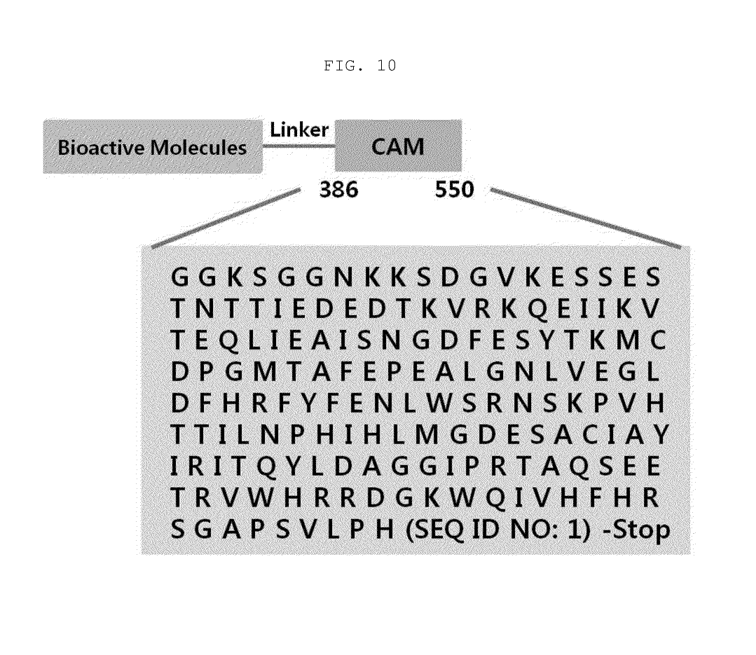

Various proteins were fused to the self-association domain (FIGS. 9 and 10) of the C-terminal end (or N-terminal end) of calcium/calmodulin-dependent kinase II (CAM) protein and used in the display and analysis of materials in the following examples. As demonstrated in the following examples, by the self-association domains of calcium/calmodulin-dependent kinase II (CAM) protein, nano-matrices and nano-assembly matrices could be formed and various bioactive materials could be displayed at high density, thereby regulating and inducing physiological functions.

In addition, the ferritin gene FTH1 (GenBank Acc. No. BC013724) and FTL(GenBank Acc. No. BC016346) was purchased from Open BioSystems (USA).

In the present invention, various proteins fused to the N-terminal end of self-association domain of ferritin (FT) protein and calcium/calmodulin-dependent kinase II (CAM) protein (FIG. 10) were used in the display and analysis of materials. Recombinant genes based on pcDNA 3.1 were constructed which can express various fusion proteins, comprising various detector proteins (e.g., FKBP and FRB) and fluorescence proteins (e.g., mRFP, EGFP, ECFP, YFP, etc.) fused to calcium/calmodulin-dependent kinase II (CAM) protein or ferritin protein (hereinafter also referred to as FT), in mammalian cells by CMV promoter. In this analysis, rapamycin was used as a material for regulating the interaction between detector materials, and mCerulean, mCherry and mCitrine were used as fluorescence proteins.

The recombinant genes FKBP-mCerulean-FT and FRB-mCitrine-CAM were introduced into previously cultured HeLa cells (ATCC No. CCL-2) using electroporation (1000 V, 35 ms, 2 pulses) or lipofectamine, and then the cells were plated on a 16-well chamber slide (Nunc) and incubated in a 5% CO₂ incubator at 37° C. for 24 hours to express the fusion proteins. For imaging, the cell culture medium was changed from 10% FBS-containing DMEM (Gibco) to OPTI-MEM (Gibco), and then the cells were treated with 250 nM rapamycin (Calbiochem) (1 mM stock concentration; in DMSO), and the distribution of the ferritin fusion protein in the cells was observed with a confocal microscope.

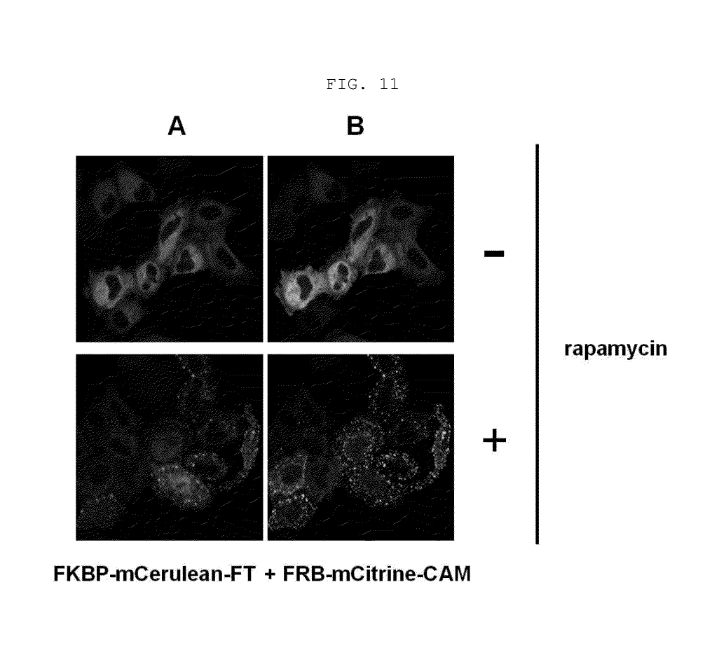

As a result, as shown in FIG. 11, the interaction between the detector materials FKBP and FRB displayed on nano-matrices was induced by treatment with the regulator material rapamycin, and thus a nano-assembly matrix that is a dotted image pattern having high signal intensity was formed according to the scheme of FIG. 5. Cells not treated with rapamycin were used as a negative control group. Thus, it was shown that, by the self-association domain of calmodulin-dependent kinase II (CAM) protein, nano-matrices and a nano-assembly matrix could be formed and bioactive materials as detector materials could be displayed at high density.

### Example 2

### Analysis of Formation of Nano-Assembly Matrix and Display of Materials by Self-Association Domain of Calcium/Calmodulin-Dependent Kinase II (CAM) Protein

As described in Example 1, various proteins fused to the N-terminal end of self-association domain of calmodulin-dependent kinase II (CAM) protein (FIG. 10) were used in the display and analysis of materials.

FKBP(F36M)2 was used as a mediator (regulator) material, and mCerulean and mCitrine were used as fluorescence proteins. According to the method described in Example 1, FKBP(F36M)2-mCerulean-CAM and FRB-mCitrine fusion proteins were expressed together in HeLa cells (ATCC No. CCL-2). Cells not treated with rapamycin were used as a negative control group. Herein, FKBP(F36M)2 is a dimeric form obtained by replacing the 36^{th} amino acid residue phenylalanine of monomeric FKBP with methionine, and as found in previous experiments, FKBP(F36M)2 can be self-associated to induce the formation of a nano-assembly matrix. In other words, it was found that the mutated FKBP functions as a mediator (regulator) material.

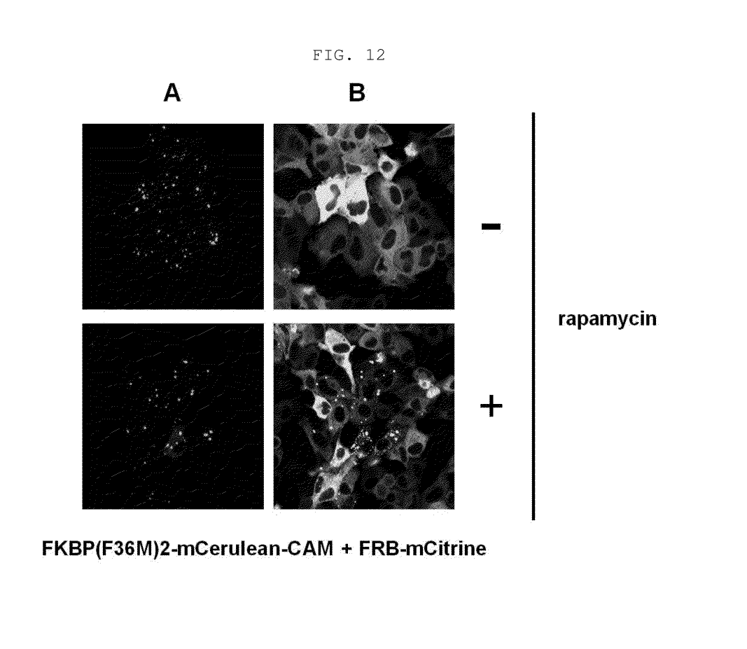

As a result, as shown in FIG. 12, when the cells were treated with the regulator material rapamycin, FKBP(F36M)2 displayed on nano-matrices was self-associated to form a nano-assembly matrix that is a dotted image pattern having high signal intensity, and then the interaction between the FKBP and FRB displayed on the nano-assembly matrix was induced, and thus FRB-mCitirine was recruited and displayed on the nano-assembly matrix according to the scheme of FIG. 3. In addition, it was shown that the mutated FKBP interacted with the detector material FRB (prey) and that the interaction between the detector materials (FKBP(F36M) and FRB) was specific for rapamycin (that mediates the interaction) and displayed on the nano-assembly matrix.

Thus, it was shown that, by the self-association domain of calmodulin-dependent kinase II (CAM) protein, a nano-assembly matrix could be formed and bioactive materials could be displayed at high density.

### Example 3

### Analysis of Formation of Nano-Assembly Matrix and Display of Materials by Ferritin (FT) Protein

According to the method described in Example 1, various proteins fused to the N-terminal end of ferritin (FT) protein were used in display and analysis. FKBP(F36M)2 was used as a mediator (regulator) material, and mCerulean and mCitrine were used as fluorescence proteins. Cells not treated with rapamycin were used as a negative control group.

Specifically, according to the method in Example 1, FKBP(F36M)2-mCherry-FT and FRB-EGFP fusion proteins were expressed together in HeLa cells (ATCC No. CCL-2).

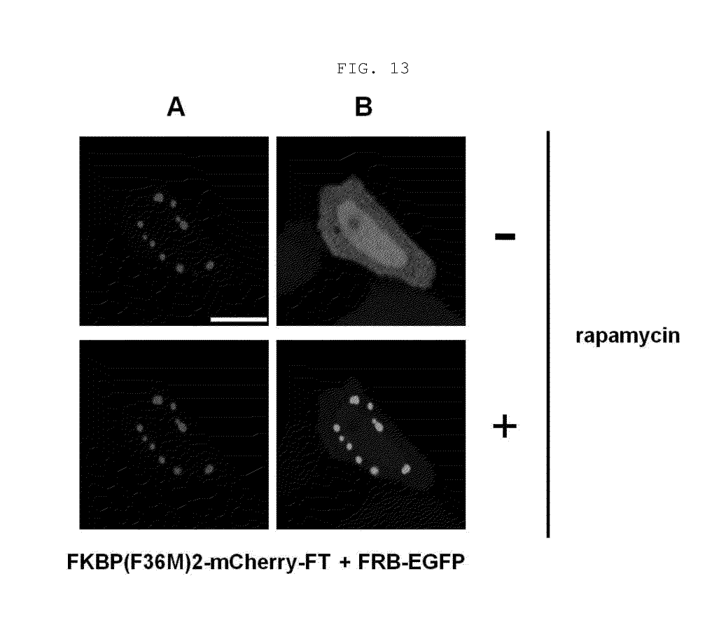

As a result, as shown in FIG. 13, when the cells were treated with 250 nM of rapamycin, FKBP(F36M)2 displayed on nano-matrices was self-associated to form a nano-assembly matrix that is a dotted image pattern having high signal intensity, and then the interaction between the FKBP and FRB displayed on the nano-assembly matrix was induced, and thus FRB-EGFP was recruited and displayed on the nano-assembly matrix according to the scheme of FIG. 3.

Thus, it was shown that, not only by CAM in Example 2, but also by ferritin (FT) protein, the nano-assembly matrix could be formed and bioactive materials could be displayed at high density.

### Example 4

### Analysis of Regulation of Intracellular Signaling and Transcriptional Activity of NFkB by Materials Displayed at High Density on Nano-Assembly Matrix Formed by Self-Association Domain of Calcium/Calmodulin-Dependent Kinase II (CAM) Protein

According to the method described in Example 1, various proteins fused to the self-association domain of calcium/calmodulin-dependent kinase II (CAM) were used in the display and analysis of materials.

Specifically, according to the method shown in Example 1, FKBP(F36M)2-mCerulean-CAM and FRB-Rel were expressed together in HeLa cells (ATCC No. CCL-2). FRB-Rel is a fusion protein of the FRB domain and the Rel domain of RelA. The cells were treated with a rapamycin analog (Clontech), and then with TNF-a. Where the cells were treated with the rapamycin analog, a T2098L mutant was used as FRB. Whether TNF-a activated the intracellular signaling and transcriptional activity of NFkB was analyzed by measuring the expression level of the reporter gene by NFkb.

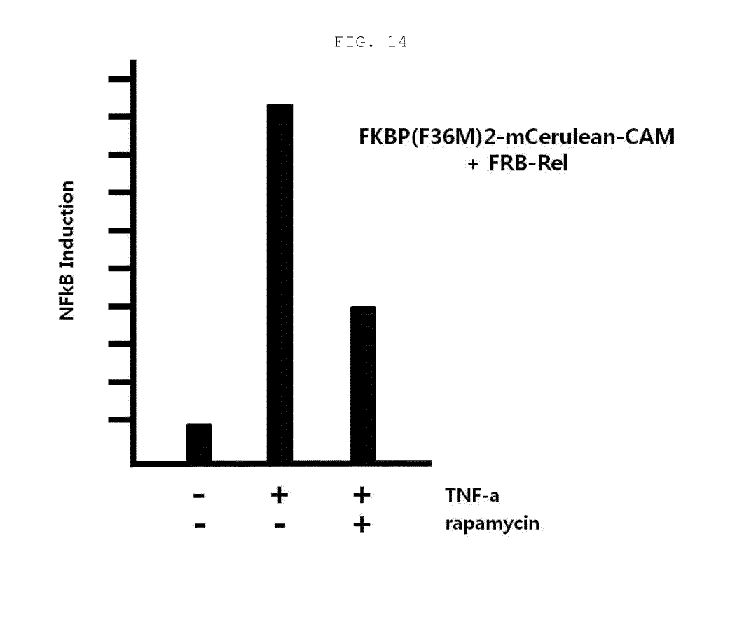

As a result, as shown in FIG. 14, when the cells were treated with the rapamycin analog, FRB-Rel was recruited and displayed on a nano-assembly matrix formed from FKBP(F36M)2-mCerulean-CAM (FIG. 12), and thus the intracellular signaling and transcriptional activity of NFkB activated by TNF-a were regulated and induced.

Thus, it was shown that, when bioactive materials are displayed at high density on a nano-assembly matrix formed by the self-association domain of calcium/calmodulin-dependent kinase II (CAM) protein according to the scheme of FIG. 3, intracellular functions can be regulated and induced.

### Example 5

### Analysis of Regulation of Intracellular Signaling and Transcriptional Activity of NFkB by Materials Displayed at High Density on Nano-Assembly Matrix Formed by Ferritin (FT) Protein

According to the method described in Example 1, various proteins fused to the N-terminal end of ferritin (FT) protein were used in the display and analysis of materials.

Specifically, according to the method described in Example 1, FKBP(F36M)2-mCherry-FT and FRB-Rel fusion proteins were expressed together in HeLa cells (ATCC No. CCL-2). The cells were treated with a rapamycin analog, and then with TNF-a. Whether TNF-a activated the intracellular signaling and transcriptional activity of NFkB was analyzed by measuring the expression level of the reporter gene by NFkb.

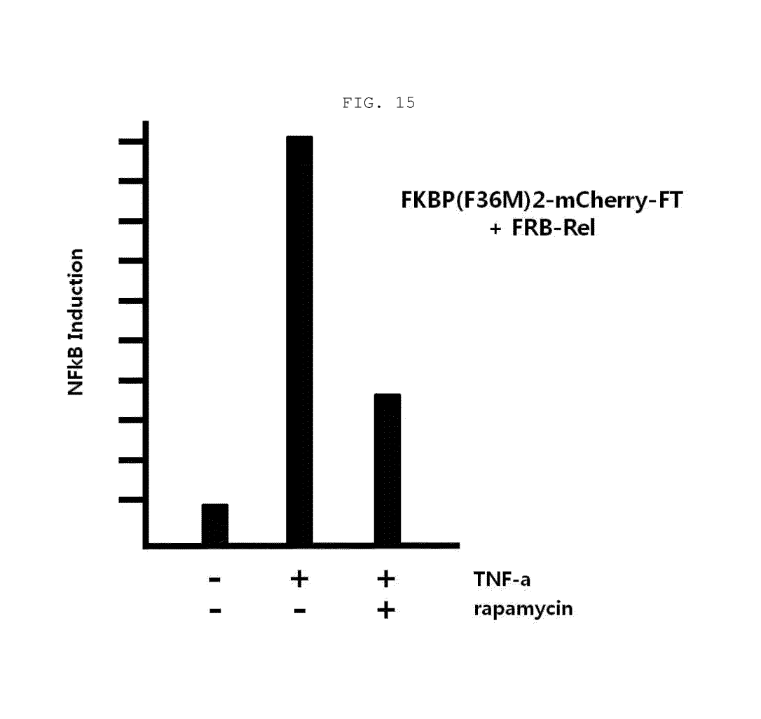

As a result, as shown in FIG. 15, as shown in FIG. 15, when the cells were treated with the rapamycin analog, FRB-Rel was recruited and displayed on a nano-assembly matrix formed from FKBP(F36M)2-mCherry-FT (FIG. 13), and thus the intracellular signaling and transcriptional activity of NFkB activated by TNF-a were regulated and induced.

Thus, it was shown that, when bioactive materials are displayed at high density on a nano-assembly matrix formed by ferritin (FT) protein according to the scheme of FIG. 3, intracellular functions can be regulated and induced.

### Example 6

### Analysis of Regulation of Intracellular Signaling and Transcriptional Activity of NFkB by Materials Displayed at High Density on Nano-Matrices Formed by Ferritin (FT) Protein

According to the method describe in Example 1, various proteins fused to the N-terminal end of ferritin (FT) protein were used in the display and analysis of materials.

Specifically, according to the method described in Example 1, a Rel-FT fusion protein was expressed in HeLa cells (ATCC No. CCL-2). The resulting cells were treated with TNF-a. Whether TNF-a activated the intracellular signaling and transcriptional activity of NFkB was analyzed by measuring the expression level of the reporter gene by NFkb.

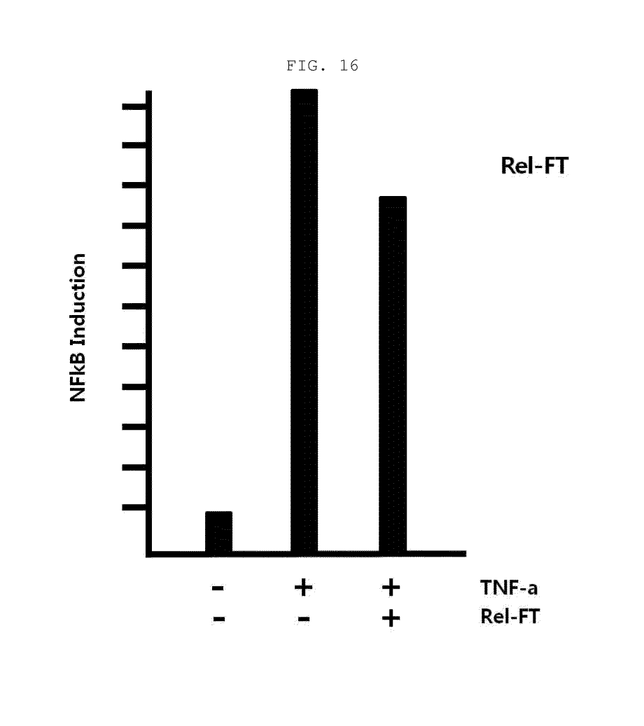

As a result, as shown in FIG. 16, Rel was displayed on the nano-matrices formed from ferritin (FT) protein, and thus the intracellular signaling and transcriptional activity of NFkB activated by TNF-a were regulated and induced. Thus, it was shown that, when bioactive materials are displayed at high density on the nano-matrices formed by ferritin (FT) protein according to the scheme of FIG. 4, intracellular functions can be regulated and induced.

### Example 7

### Analysis of Regulation of Intracellular Signaling and Transcriptional Activity of NFkB by Materials Displayed at High Density on Nano-Assembly Matrix Formed by Ferritin (FT) Protein

According to the method describe in Example 1, various proteins fused to the N-terminal end of ferritin (FT) protein were used in the display and analysis of materials.

Specifically, according to the method described in Example 1, Rel-FT, FKBP-FT and FRB-FT fusion proteins were expressed in HeLa cells (ATCC No. CCL-2). The resulting cells were treated with a rapamycin analog, and then with TNF-a. Whether TNF-a activated the intracellular signaling and transcriptional activity of NFkB was analyzed by measuring the expression level of the reporter gene by NFkb.

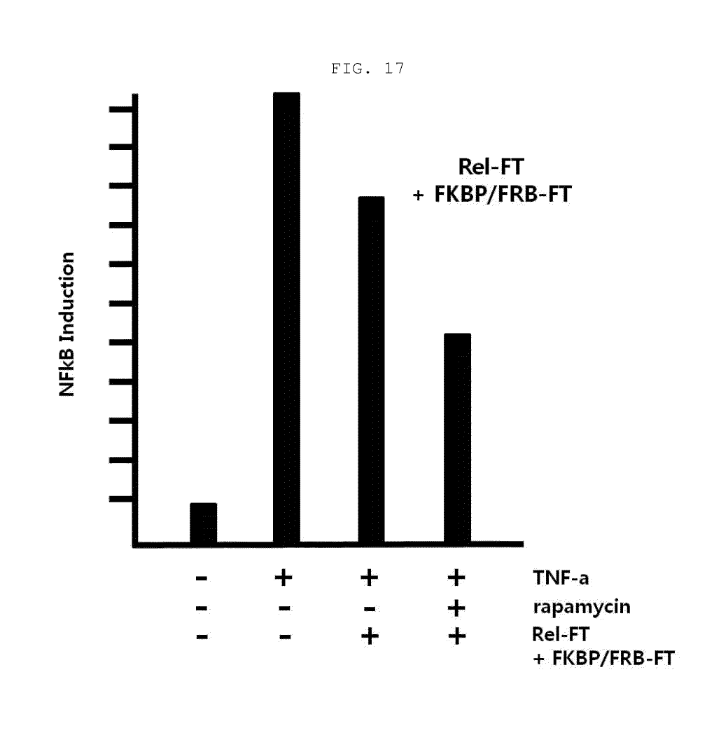

As a result, as shown in FIG. 17, when the cells were treated with the rapamycin analog, Rel-FT was displayed on the nano-assembly matrix formed from FKBP-FT and FRB-FT, and thus the intracellular signaling and transcriptional activity of NFkB activated by TNF-a were regulated and induced. Thus, it was shown that, when bioactive materials are displayed at high density on the nano-assembly matrix formed by ferritin (FT) protein according to the scheme of FIG. 2, intracellular functions can be regulated and induced.

### Example 8

### Analysis of Regulation of Intracellular Signaling and Transcriptional Activity of NFkB by Materials Displayed at High Density on Nano-Matrices Formed by Self-Association Domain of Calcium/Calmodulin-Dependent Kinase II (CAM) Protein

According to the method describe in Example 1, various proteins fused to the N-terminal end of self-association domain of calcium/calmodulin-dependent kinase II (CAM) protein were used in the display and analysis of materials.

Specifically, according to the method described in Example 1, a FRB-Rel-CAM fusion protein was expressed in HeLa cells (ATCC No. CCL-2). The resulting cells were treated with TNF-a. Whether TNF-a activated the intracellular signaling and transcriptional activity of NFkB was analyzed by measuring the expression level of the reporter gene by NFkb.

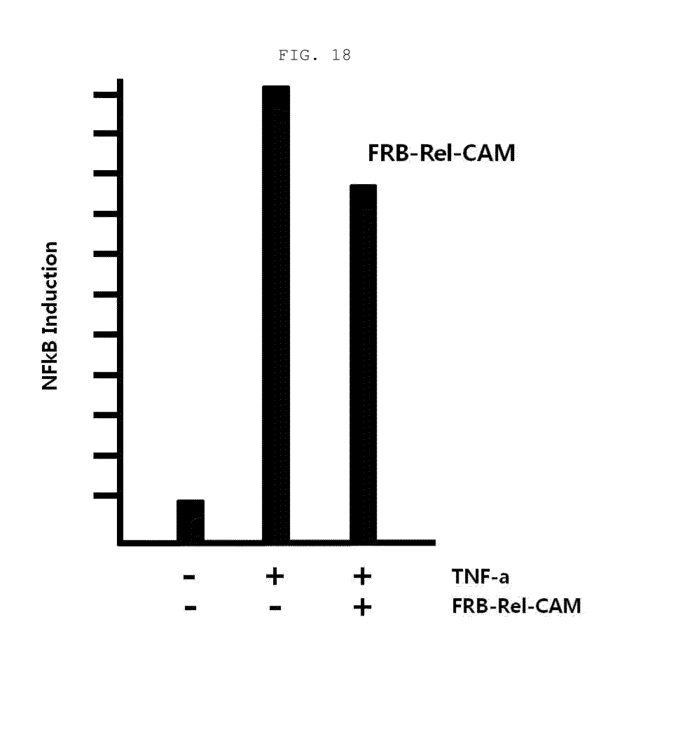

As a result, as shown in FIG. 18, Rel was displayed on the nano-matrices formed from the self-association domain of calcium/calmodulin-dependent kinase II (CAM) protein, and thus the intracellular signaling and transcriptional activity of NFkB activated by TNF-a were regulated and induced. Thus, it was shown that, when bioactive materials are displayed at high density on the nano-matrices formed by the self-association domain of calcium/calmodulin-dependent kinase II (CAM) protein according to the scheme of FIG. 4, intracellular functions can be regulated and induced.

### Example 9

### Analysis of Regulation of Intracellular Signaling and Transcriptional Activity of NFkB by Materials Displayed at High Density on Nano-Assembly Matrix Formed by Self-Association Domain of Calcium/Calmodulin-Dependent Kinase II (CAM) Protein and Ferritin (FT) Protein

According to the method described in Example 1, various proteins fused to the N-terminal end of self-association domain of calcium/calmodulin-dependent kinase II (CAM) protein were used in the display and analysis of materials.

Specifically, according to the method described in Example 1, FRB-Rel-CAM and FKBP-FT fusion proteins were expressed in HeLa cells (ATCC No. CCL-2). The resulting cells were treated with a rapamycin analog, and then with TNF-a. Whether TNF-a activated the intracellular signaling and transcriptional activity of NFkB was analyzed by measuring the expression level of the reporter gene by NFkb.

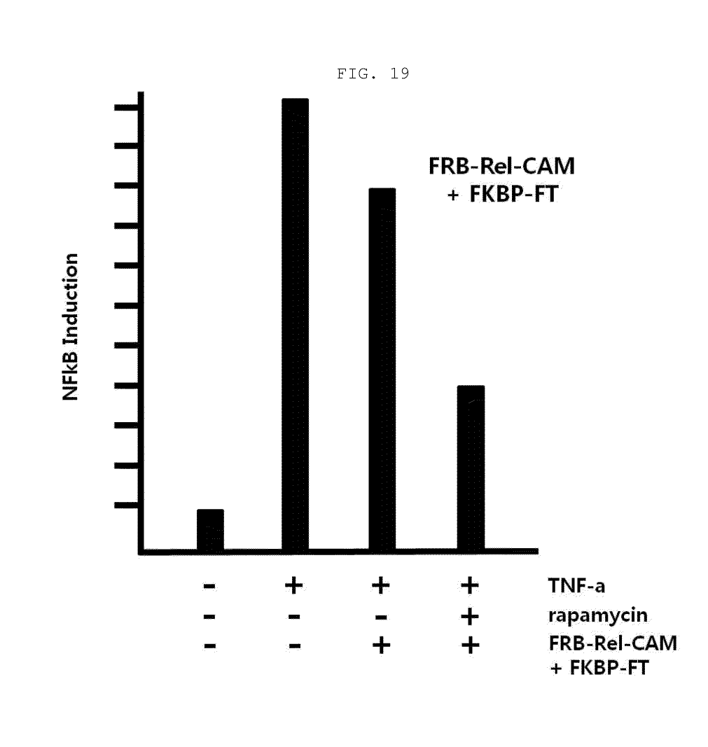

As a result, as shown in FIG. 19, when the cells were treated with the rapamycin analog, Rel was displayed on the nano-assembly matrix formed from FRB-Rel-CAM and FKBP-FT, and thus the intracellular signaling and transcriptional activity of NFkB activated by TNF-a were regulated and induced. Thus, it was shown that, when bioactive materials are displayed at high density on the nano-assembly matrix formed by the self-association domain of calcium/calmodulin-dependent kinase II (CAM) protein and ferritin (FT) protein according to the scheme of FIG. 5, intracellular functions can be regulated and induced.

### Example 10

### Isolation and Purification of Materials Displayed at High Density on Nano-Assembly Matrix Formed by Self-Association Domain of Calcium/Calmodulin-Dependent Kinase II (CAM) Protein and Ferritin (FT) Protein

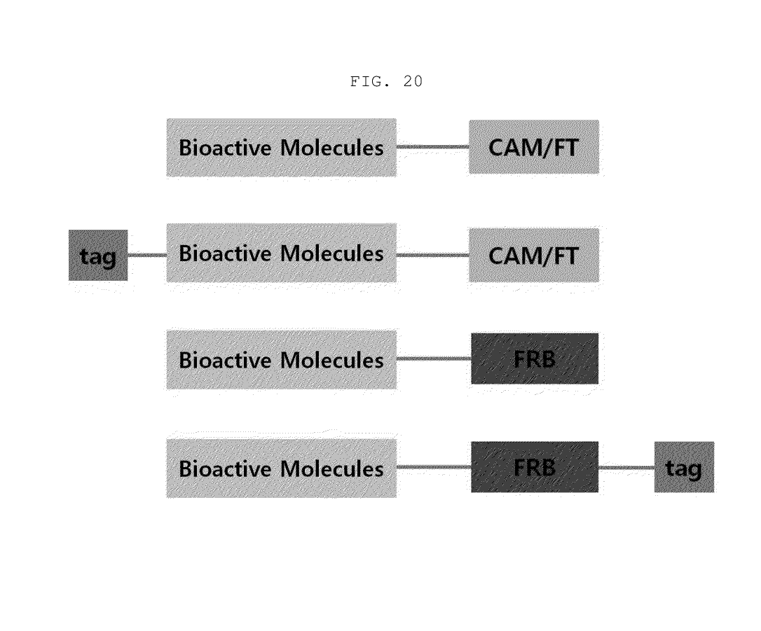

Various proteins fused to the N-terminal end (or C-terminal end) of self-association domain of calcium/calmodulin-dependent kinase II (CAM) protein and ferritin (FT) protein were used in the high-density display of materials through various methods, including a direct method or an indirect method comprising fusion to the FRB domain (FIG. 20).

The fusion proteins thus displayed were isolated and purified, and the bioactive materials were treated by high-density display outside cells or in vivo. As a result, it was found that the local concentration of the bioactive materials effectively increases, and thus they can induce physiological regulation in cells and in vivo.

### Example 11

### Various Therapeutic and Diagnostic Materials Displayed at High Density on Nano-Assembly Matrix Formed by Self-Association Domain of Calcium/Calmodulin-Dependent Kinase II (CAM) Protein and Ferritin (FT) Protein

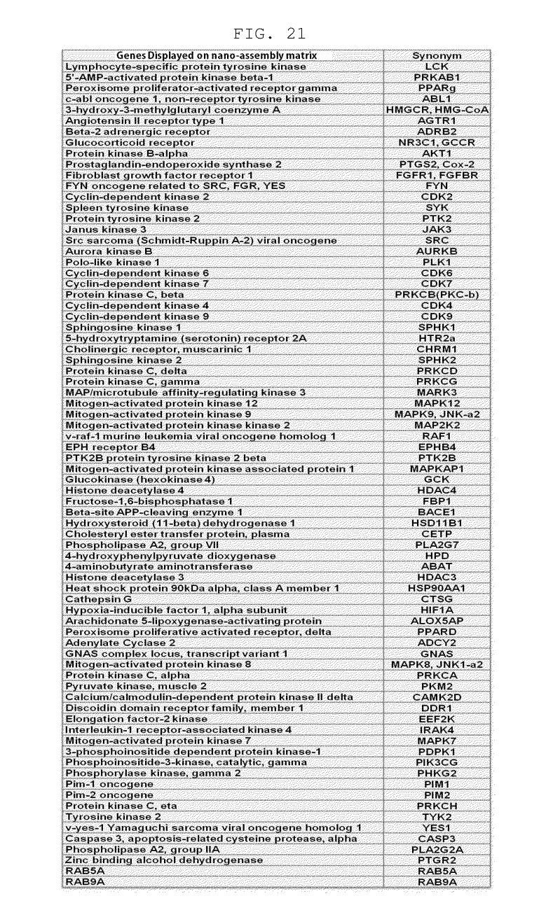

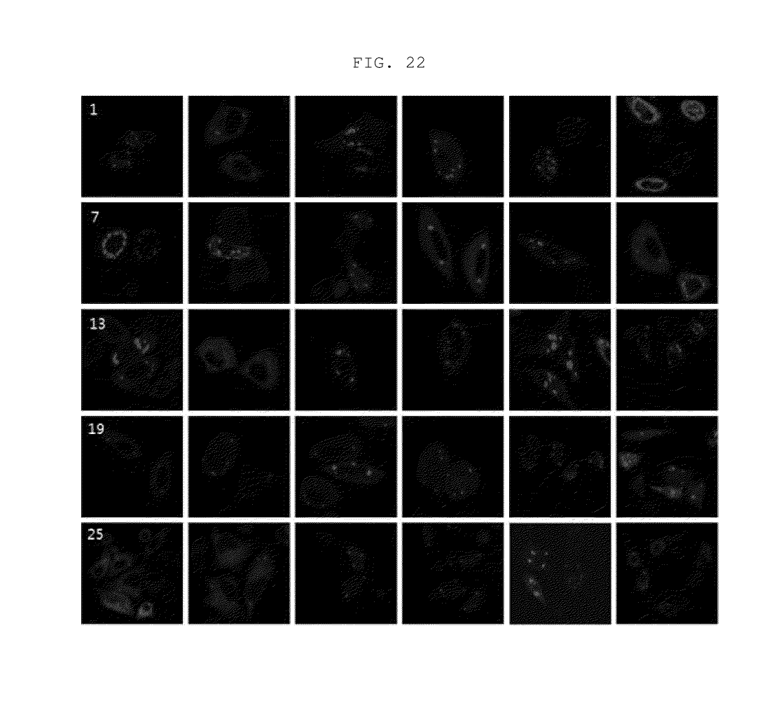

FIG. 21 shows examples of various therapeutic and diagnostic proteins fused to the self-association domain of calcium/calmodulin-dependent kinase II (CAM) protein and ferritin (FT) protein so as to be able to induce physiological regulation in cells and in vivo. Some examples of such therapeutic and diagnostic proteins were fused to FRB-mCherry and expressed in cells which were then with rapamycin. As a result, as shown in FIG. 22, these proteins could be displayed at high density on the nano-assembly matrix formed by the self-association domain of calcium/calmodulin-dependent kinase II (CAM) protein and ferritin (FT) protein to which FKBP(F36M)2 was fused, as shown in FIGS. 12 and 13.

### INDUSTRIAL APPLICABILITY

As described above, according to the present invention, one or more bioactive materials can be displayed at high density on an artificial nano-assembly matrix in the same field or system in vitro and in vivo, and thus physiological functions that are mediated by these bioactive materials can be effectively regulated. For example, with respect to pharmacological activities, when bioactive materials related to treatment and diagnosis are displayed on nano-assembly matrices, the possibility of their interaction with the targets related to pharmacological activities and diagnosis as known in the results of previous research and development will increase, pharmacokinetics and biodistribution will be improved, resulting in an increase in efficacy, suggesting that physiological functions can be effectively regulated.

In addition, the assembly and disassembly of the nano-assembly matrix or the display or trapping of specific materials on the nano-assembly matrix can be artificially regulated, and thus physiological functions in cells or in vivo can be optionally regulated and induced.

Therefore, according to the present invention, various physiological functions that are mediated by specific bioactive materials in cells or laving bodies can be effectively regulated and induced in vitro or in vivo.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for regulating or inducing physiological conditions or functions in cells or in vivo, the method comprising the steps:
(i) providing mediator (regulator) materials, detector materials and a nano-assembly matrix-forming material to the same field or system; and
(ii) forming a nano-assembly unit matrix and/or nano-assembly matrix by the interaction between the mediator (regulator) materials; the mediator (regulator) materials and the detector materials; the detector materials; the nano-assembly matrix-forming materials; the mediator (regulator) materials and the nano-assembly forming materials; and/or the detector materials and the nano-assembly forming materials to display the mediator (regulator) materials and/or the detector materials at high density, thereby regulating or inducing physiological conditions or functions that are mediated by the mediator (regulator) materials and/or the detector materials.

2.-3. (canceled)

4. The method of claim 1, wherein in step (i), a material that mediates or regulates the interaction between the mediator (regulator) materials; the mediator (regulator) materials and the detector materials; the detector materials; the nano-assembly matrix-forming materials; the mediator (regulator) materials and the nano-assembly forming materials; and/or the detector materials and the nano-assembly forming materials to display the mediator (regulator) materials and/or the detector materials is additionally added.

5.-6. (canceled)

7. The method of claim 1, wherein the interaction between the detector materials; the mediator (regulator) materials and the detector materials; the detector materials; the nano-assembly matrix-forming materials; the mediator (regulator) materials and the nano-assembly forming materials; and/or the detector materials and the nano-assembly forming materials to display the mediator (regulator) materials and/or the detector materials occurs directly or indirectly.

8. The method of claim 1, wherein one or more mediator (regulator) materials that mediate (regulate) the interaction between the detector materials or between the detector materials and the nano-assembly matrix-forming materials are additionally added.

9. The method of claim 8, wherein the mediator (regulator) materials are added with the mediator (regulator) materials fused to the detector materials.

10. The method of claim 1, wherein the detector materials, mediator (regulator) materials or the nano-assembly matrix-forming materials are labeled with a label.

11. The method of claim 10, wherein the label includes magnetic materials, radioactive materials, enzymatic materials for ELISA, fluorescent materials, and luminescent materials.

12. The method of claim 11, wherein the fluorescent materials include fluorescent dyes, fluorescent proteins and fluorescent nanoparticles.

13. The method of claim 1, wherein the detector materials and the mediator (regulator) materials are bioactive molecules.

14. The method of claim 13, wherein the bioactive molecules are one or more selected from the group consisting of nucleic acids, nucleotides, proteins, peptides, amino acids, saccharides, lipids, vitamins, and chemical compounds.

15. The method of claim 1, wherein the nano-assembly matrix-forming materials are poly/multi-valent materials that have a plurality of the same or different binding moieties and can form matrices by the interaction or self-assembly between them.

16. The method of claim 15, wherein the nano-assembly matrix-forming materials are selected from the group consisting of proteins having self-assembly or self-association domains, gold nanoparticles, Q dots, and magnetic nanoparticles.

17. The method of claim 16, wherein the proteins having self-assembly or self-association domains are selected from the group consisting of ferritin, ferritin-like protein, DPS (DNA binding protein from starved cells), DPS-like protein, HSP (heat shock protein), magnetosome protein, viral protein, calcium/calmodulin-dependent kinase II, and dsRed.

18. The method of claim 1, wherein the method is performed in a cell, a tissue or a living body.

19. The method of claim 18, wherein the method is performed in the living cells or tissues of Zebra fish, C. elegans, yeast, flies or frogs, mammals, and plants.

20. The method of claim 18, wherein the introduction of the materials into the cell, the tissue or the living body is performed by any one method selected from the group consisting of direct injection, a method employing a transducible peptide, a fusogenic peptide, a lipid delivery system or a combination thereof, electroporation, magnetofection, and parenteral administration, oral administration, intranasal administration, subcutaneous administration, aerosolized administration and intravenous administration into mammals including humans.

21. The method of claim 1, wherein the formation of the nano-assembly matrix is measured by any one selected from the group consisting of a magnetic method, a radioactive method, a method employing an enzyme for ELISA, a method of detecting a fluorescent or luminescent material, an optical method, or a method employing a microscope, an imaging system, a scanner, a reader, a spectrophotometer, MRI (magnetic resonance imaging), SQUID, an MR relaxometer, flow Cytometry, FACS (fluorescene associated cell sorting), a fluorometer or a luminometer.

22. The method of claim 1, wherein regulator molecules are loaded at high density either into the nano-assembly unit matrix or the nano-assembly matrix, and physiological activities or functions are regulated or induced by the loaded molecules that are exposed as a result of the dis-assembly of the nano-assembly unit matrix or the nano-assembly matrix.

23. A composition for material delivery, vaccination, prevention, or treatment against disease related to physiological conditions or functions in cells or in vivo, the composition comprising a nano-assembly unit matrix and/or nano-assembly matrix isolated by a method comprising the steps of:
(i) providing mediator (regulator) materials, detector materials and a nano-assembly matrix-forming material to the same field or system; and
(ii) forming a nano-assembly unit matrix and/or nano-assembly matrix by the interaction between the mediator (regulator) materials; the mediator (regulator) materials and the detector materials; the detector materials; the nano-assembly matrix-forming materials; the mediator (regulator) materials and the nano-assembly forming materials; the detector materials and the nano-assembly forming materials to display the detector materials at high density, and isolating the formed nano-assembly unit matrix and/or nano-assembly matrix.

24.-28. (canceled)

29. The method of claim 23, wherein regulator materials capable of interacting with the displayed detector materials are additionally provided and displayed at high density, followed by isolation.

30. The method of claim 23, wherein regulator molecules are loaded at high density either into the nano-assembly unit matrix or the nano-assembly matrix.

31. The method of claim 23, wherein the detector materials, mediator (regulator) materials or the nano-assembly matrix-forming materials are labeled with a label.

32. A method for screening a material that regulates or induces physiological conditions or functions in cells or in vivo, the method comprising the steps of:
(i) providing mediator (regulator) materials, detector materials and a nano-assembly matrix-forming material to the same field or system;
(ii) forming a nano-assembly unit matrix and/or nano-assembly matrix by the interaction between the mediator (regulator) materials; the mediator (regulator) materials and the detector materials; the detector materials; the nano-assembly matrix-forming materials; the mediator (regulator) materials and the nano-assembly forming materials; and/or the detector materials and the nano-assembly forming materials to display the detector materials at high density;
(iii) providing target candidates to the nano-assembly unit matrix and/or nano-assembly matrix; and
(iv) selecting, as the material that regulates or induces physiological conditions or functions in cells or in vivo, a target candidate corresponding to a case in which physiological conditions or functions in the presence of the target candidate change compared to physiological conditions or functions in the absence of the target candidate.

33.-36. (canceled)

37. The method of claim 32, wherein regulator materials capable of interacting with the displayed detector materials are additionally provided and displayed at high density.

38. The method of claim 32, wherein the detector materials, mediator (regulator) materials or the nano-assembly matrix-forming materials are labeled with a label.
